SEQUENCE LISTING (69 sequences)

SEQ ID NO: 1 - DNA, 1119 bp, synthetic construct
  misc_feature 1..1119: note = Laboratory made
  source 1..1119: mol_type = other DNA
atgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccatag

SEQ ID NO: 2 - DNA, 405 bp, synthetic construct
  misc_feature 1..405: note = Laboratory made
  source 1..405: mol_type = other DNA
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatag

SEQ ID NO: 3 - DNA, 1178 bp, synthetic construct
  misc_feature 1..1178: note = laboratory made
  source 1..1178: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 4 - DNA, 30 bp, synthetic construct
  misc_feature 1..30: note = laboratory made
  source 1..30: mol_type = other DNA
ggaggaaaaactgtttcatacagaaggcgt

SEQ ID NO: 5 - DNA, 32 bp, synthetic construct
  misc_feature 1..32: note = laboratory made
  source 1..32: mol_type = other DNA
tagagggtatataatggaagctcgaattccag

SEQ ID NO: 6 - DNA, 320 bp, synthetic construct
  misc_feature 1..320: note = laboratory made
  source 1..320: mol_type = other DNA
aataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcggaggaaaaactgtttcatacagaaggcgtggaggaaaaactgtttcatacagaaggcgtggaggaaaaactgtttcatacagaaggcgtagatctagactctagagggtatataatggaagctcgaattccag

SEQ ID NO: 7 - DNA, 6887 bp, synthetic construct
  misc_feature 1..6887: note = laboratory made
  source 1..6887: mol_type = other DNA
aggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggtacctctagagctgaccaaaagagcaccaaaggcgccctgaccttcagcccctacctgcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgataatacgactcactatagggagacccaagctggaattcggcggccgccaccatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttaatgcataacttcgtataatgtatgctatacgaagttatgttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagtgtaacgccaccatgaccgagtacaagcctaccgtgaggctggccacccgggacgacgtgcccagagccgtgaggacactggccgccgccttcgccgactaccccgccacccggcacaccgtggaccccgaccggcacatcgagcgggtgaccgagctgcaggaactgttcctgaccagagtgggcctggatatcggcaaagtgtgggtggccgacgacggagccgccgtggccgtgtggaccacccccgagtccgtggaggccggagccgtgtttgccgagatcggccccaggatggccgagctgtccggcagcaggctggccgcccagcagcagatggaaggcctgctggcccctcaccggcccaaagagcccgcctggttcctggccaccgtgggcgtgagccccgaccaccagggcaagggcctgggcagcgccgtggtgctgccaggcgtggaagccgccgagagggccggagtgcccgccttcctggaaaccagcgcccccaggaacctgcccttctacgagcggctgggctttaccgtgaccgccgacgtggaggtgccagagggccccaggacctggtgcatgacccggaagccaggcgcctgagaaaagcttataacttcgtataatgtatgctatacgaagttataacttgtttattgcagcttataatggttacaaataaagcaatagcatcacaaatttcacaaataaagcatttttttcactgcattctagttgtggtttgtccaaactcatcaatgtatcttatcatgtctgtgcggtgggctctatggcttctgaggcggaaagaaccagctggggctctagggggtatcccctctagagtactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagact

SEQ ID NO: 8 - DNA, 2497 bp, synthetic construct
  misc_feature 1..2497: note = laboratory made
  source 1..2497: mol_type = other DNA
ataacttcgtataatgtatgctatacgaagttatggcgcgccgaagttcctattcttctagaagaataggaacttccgaataggaacttcctgcacgtgaacttgtttattgcagcttataatggttacaaataaagcaatagcatcacaaatttcacaaataaagcatttttttcactgcattctagttgtggtttgtccaaactcatcaatgtatcttatcatgtctgtgagctgaaggtacgctgtatctcagtcagtcaagctagctcaggtttagttcctggtgtacttgagggggatgagttcctcaatggtggttttgaccagcttgccattcatctcaatgagcacaaagcagtcaggagcatagtcagagatcagctctctacacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcttcaatgtggacagcagagatgatctccccagtcttggtcctgatggccgccccgacatggtgcttgttgtcctcatagagcatggtgatcttctcagtggcgacctccaccagctccagatcctgctgagagatgttgaaggttttcatgttgggatccacgtggagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtgaagttcctattctctagaaagaataggaacttccgaataggaacttcggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcggaggaaaaactgtttcatacagaaggcgtggaggaaaaactgtttcatacagaaggcgtggaggaaaaactgtttcatacagaaggcgtagatctagactctagagggtatataatggaagctcgaattccagcttggcattccggtactgttggtaaaaagcttggcaatccggtactgcctgcaggaccgccatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagaccccttaagaattcgcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttaatgcataacttcgtataatgtatgctatacgaagttat

SEQ ID NO: 9 - DNA, 1211 bp, synthetic construct
  misc_feature 1..1211: note = laboratory made
  source 1..1211: mol_type = other DNA
ggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 10 - DNA, 500 bp, synthetic construct
  misc_feature 1..500: note = laboratory made
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctacccctcccctagtcaggaagttcccccccgccccgcagctcgcgtcatgcaggacgtgacaaatggaagtagcacgtctcactagtctcgtgcaaatggacagcaccgctgagcaatggaagcgggtaggcccttggggcagcggccaatagcagctttgctccttcgctttctgggctcagaggctgggaaggggtgggtccgggggcgggctcaggggcgggctcaggggcggggcgggcgcccgaaggtcctccggaggcccggcattccgcacgcttcaaaagcgcacgtctgccgcgctgttctcttcttcctcatctccgggcctttcg

SEQ ID NO: 11 - DNA, 508 bp, synthetic construct
  misc_feature 1..508: note = laboratory made
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 12 - protein, 254 aa, synthetic construct
  REGION 1..254: note = laboratory made
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 13 - DNA, 765 bp, synthetic construct
  misc_feature 1..765: note = laboratory made
  source 1..765: mol_type = other DNA
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 14 - DNA, 483 bp, synthetic construct
  misc_feature 1..483: note = laboratory made
  source 1..483: mol_type = other DNA
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 15 - protein, 164 aa, synthetic construct
  REGION 1..164: note = laboratory made
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDELKDEL

SEQ ID NO: 16 - DNA, 297 bp, synthetic construct
  misc_feature 1..297: note = laboratory made
  source 1..297: mol_type = other DNA
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaa

SEQ ID NO: 17 - DNA, 60 bp, synthetic construct
  misc_feature 1..60: note = laboratory made
  source 1..60: mol_type = other DNA
cagtgcaccaactacgccctgctgaagctggccggcgacgtggagagcaaccctggccct

SEQ ID NO: 18 - DNA, 54 bp, synthetic construct
  misc_feature 1..54: note = laboratory made
  source 1..54: mol_type = other DNA
gagggcagaggcagcctgctgacctgcggcgatgtggaggaaaacccaggccca

SEQ ID NO: 19 - DNA, 806 bp, synthetic construct
  misc_feature 1..806: note = laboratory made
  source 1..806: mol_type = other DNA
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggtac

SEQ ID NO: 20 - DNA, 1178 bp, synthetic construct; identical to SEQ ID NO: 3
  misc_feature 1..1178: note = Laboratory made
  source 1..1178: mol_type = other DNA

SEQ ID NO: 21 - DNA, 19 bp, synthetic construct
  misc_feature 1..19: note = laboratory made
  source 1..19: mol_type = other DNA
taatacgactcactatagg

SEQ ID NO: 22 - DNA, 1116 bp, synthetic construct
  misc_feature 1..1116: note = laboratory made
  source 1..1116: mol_type = other DNA
atgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctcccca

SEQ ID NO: 23 - DNA, 66 bp, synthetic construct
  misc_feature 1..66: note = laboratory made
  source 1..66: mol_type = other DNA
ggatctggagctactaacttcagcctgctgaagcaggctggagacgtggaggagaaccctggacct

SEQ ID NO: 24 - DNA, 69 bp, synthetic construct
  misc_feature 1..69: note = laboratory made
  source 1..69: mol_type = other DNA
atggactggatctggcggattctgtttctcgtgggagctgccacaggcgctcattctgctcagcctgcc

SEQ ID NO: 25 - DNA, 1152 bp, synthetic construct
  misc_feature 1..1152: note = laboratory made
  source 1..1152: mol_type = other DNA
gatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 26 - DNA, 765 bp, synthetic construct; identical to SEQ ID NO: 13
  misc_feature 1..765: note = laboratory made
  source 1..765: mol_type = other DNA

SEQ ID NO: 27 - DNA, 597 bp, synthetic construct
  misc_feature 1..597: note = laboratory made
  source 1..597: mol_type = other DNA
aattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgc

SEQ ID NO: 28 - DNA, 483 bp, synthetic construct; identical to SEQ ID NO: 14
  misc_feature 1..483: note = laboratory made
  source 1..483: mol_type = other DNA

SEQ ID NO: 29 - DNA, 252 bp, synthetic construct
  misc_feature 1..252: note = laboratory made
  source 1..252: mol_type = other DNA
acgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 30 - DNA, 844 bp, synthetic construct
  misc_feature 1..844: note = laboratory made
  source 1..844: mol_type = other DNA
ctagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactg

SEQ ID NO: 31 - protein, 41 aa, synthetic construct
  REGION 1..41: note = laboratory made
LKIQVRKAAITSYEKSDGVYTGLSTRNQETYETLKHEKPPQ

SEQ ID NO: 32 - DNA, 123 bp, synthetic construct
  misc_feature 1..123: note = laboratory made
  source 1..123: mol_type = other DNA
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 33 - protein, 64 aa, synthetic construct
  REGION 1..64: note = laboratory made
LSNSIMYFSHFVPVFLPAKPTTTPAPRPPTPAPTIASQPLSLRPEACRPAAGGAVHTRGLDFAC

SEQ ID NO: 34 - DNA, 192 bp, synthetic construct
  misc_feature 1..192: note = laboratory made
  source 1..192: mol_type = other DNA
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgc

SEQ ID NO: 35 - protein, 28 aa, synthetic construct
  REGION 1..28: note = laboratory made
FWVLVVVGGVLACYSLLVTVAFIIFWVR

SEQ ID NO: 36 - DNA, 84 bp, synthetic construct
  misc_feature 1..84: note = laboratory made
  source 1..84: mol_type = other DNA
ttttgggtgctggtggtcgtgggcggagtgctggcttgttattctctgctggtcaccgtggccttcatcatcttttgggtccga

SEQ ID NO: 37 - protein, 133 aa, synthetic construct
  REGION 1..133: note = laboratory made
LSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 38 - DNA, 399 bp, synthetic construct
  misc_feature 1..399: note = laboratory made
  source 1..399: mol_type = other DNA
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 39 - protein, 252 aa, synthetic construct
  REGION 1..252: note = laboratory made
AQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 40 - protein, 113 aa, synthetic construct
  REGION 1..113: note = laboratory made
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - DNA, 1410 bp, synthetic construct
  misc_feature 1..1410: note = laboratory made
  source 1..1410: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcaaggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgagtgaagttcagcagatccgccgatgcccctgcttaccagcagggccagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 42 - protein, 153 aa, synthetic construct
  REGION 1..153: note = laboratory made
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - protein, 160 aa, synthetic construct
  REGION 1..160: note = laboratory made
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 44 - protein, 372 aa, synthetic construct
  REGION 1..372: note = laboratory made
MKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 45 - protein, 98 aa, synthetic construct
  REGION 1..98: note = laboratory made
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 46 - protein, 134 aa, synthetic construct
  REGION 1..134: note = laboratory made
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 47 - DNA, 1083 bp, synthetic construct
  misc_feature 1..1083: note = laboratory made
  source 1..1083: mol_type = other DNA
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaa

SEQ ID NO: 48 - protein, 360 aa, synthetic construct
  REGION 1..360: note = laboratory made
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 49 - DNA, 336 bp, synthetic construct
  misc_feature 1..336: note = laboratory made
  source 1..336: mol_type = other DNA
atgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 50 - protein, 111 aa, synthetic construct
  REGION 1..111: note = laboratory made
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 51 - DNA, 1119 bp, synthetic construct
  misc_feature 1..1119: note = laboratory made
  source 1..1119: mol_type = other DNA
atgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaa

SEQ ID NO: 52 - protein, 372 aa, synthetic construct [sequence omitted]

SEQ ID NO: 53 - DNA, 300 bp, synthetic construct
  misc_feature 1..300: note = laboratory made
  source 1..300: mol_type = other DNA
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 54 - protein, 99 aa, synthetic construct
  REGION 1..99: note = laboratory made
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 55 - DNA, 324 bp, synthetic construct
  misc_feature 1..324: note = laboratory made
  source 1..324: mol_type = other DNA
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactga

SEQ ID NO: 56 - protein, 107 aa, synthetic construct
  REGION 1..107: note = laboratory made
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 57 - DNA, 762 bp, synthetic construct
  misc_feature 1..762: note = laboratory made
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 58 - protein, 253 aa, synthetic construct
  REGION 1..253: note = laboratory made
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - DNA, 987 bp, synthetic construct
  misc_feature 1..987: note = laboratory made
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 60 - protein, 328 aa, synthetic construct
  REGION 1..328: note = laboratory made
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 61 - protein, 536 aa, synthetic construct
  REGION 1..536: note = laboratory made
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 62 - DNA, 1611 bp, synthetic construct
  misc_feature 1..1611: note = laboratory made
  source 1..1611: mol_type = other DNA
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 63 - DNA, 477 bp, synthetic construct
  misc_feature 1..477: note = laboratory made
  source 1..477: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 64 - protein, 159 aa, synthetic construct
  REGION 1..159: note = laboratory made
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 65 - DNA, 933 bp, synthetic construct
  misc_feature 1..933: note = laboratory made
  source 1..933: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggaggaagtggaggaggtggaagtggtggtggaggtagtacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 66 - protein, 311 aa, synthetic construct
  REGION 1..311: note = laboratory made
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 67 - DNA, 510 bp, synthetic construct
  misc_feature 1..510: note = Laboratory made
  source 1..510: mol_type = other DNA
atgcgcatcagcaagccccacctgcgcagcatcagcatccagtgctacctgtgcctgctgctgaacagccacttcctgaccgaggccggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggctccgagaaggacgagctgtaa

SEQ ID NO: 68 - DNA, 1215 bp, synthetic construct
  misc_feature 1..1215: note = Laboratory made
  source 1..1215: mol_type = other DNA
atggactggatctggcggattctgtttctcgtgggagctgccacaggcgctcattctgctcagcctgccaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 69 - protein, 405 aa, synthetic construct
  REGION 1..405: note = Laboratory made
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

(19) United States
(12) Patent Application Publication
(10) Pub. No.: US 2024/0252540 A1
(43) Pub. Date: Aug. 1, 2024

(54) Cell-mediated transient delivery of immune-enhancing molecules into the tumor microenvironment

(71) Applicant: ImmunityBio, Inc., Culver City, CA (US)

(72) Inventors: John H. Lee, Culver City, CA (US); Laurent H. Boissel, Culver City, CA (US); Hans G. Klingemann, Culver City, CA (US)

(73) Assignee: ImmunityBio, Inc., Culver City, CA (US)

(21) Appl. No.: 18/610,961

(22) Filed: Mar. 20, 2024

Related U.S. Application Data
(62) Division of application No. 16/953,879, filed on Nov. 20, 2020, now Pat. No. 11,992,504.
(60) Provisional application No. 62/938,201, filed on Nov. 20, 2019.

(51) Int. Cl. A61K 35/17; C07K 14/735; C07K 16/28; C12N 5/0783

(52) U.S. Cl. CPC A61K 35/17; C07K 14/70535; C07K 16/2827; C12N 5/0646

(57) ABSTRACT

A recombinant natural killer (NK) cell or T-cell composition is transfected with a nucleic acid encoding i) a homing receptor; ii) an antigen binding protein (ABP) or a chimeric antigen receptor (CAR) that specifically binds a target antigen; iii) an Fc Receptor; and/or iv) a secreted immune modulator selected from a TGFβ inhibitor and/or IL-12, where the recombinant cell is gamma (γ)-irradiated conferring inhibition of cell proliferation with transient activity of the transfected molecules including the secreted immune modulators for up to 72 hours.

This application is a divisional application of allowed U.S. application Ser. No. 16/953,879, filed on Nov. 20, 2020, which claims priority to and the benefit of U.S. Provisional Application No. 62/938,201 filed on Nov. 20, 2019, each of which are incorporated herein by reference.

### INCORPORATION BY REFERENCE

The instant application contains a Sequence Listing which has been submitted in XML format via Patent Center and is hereby incorporated by reference in its entirety. Said XML copy was created on Mar. 7, 2024, is named 104077_0017 US2.xml, and is 125 Kbyte in size.

### FIELD OF THE INVENTION

The field of the invention is the targeting of engineered cells using the cytotoxic activated Natural Killer cell line (NK-92) as the basis to improve immunotherapies to cancer and tumors, and in particular irradiated cells for transient presence of immune-enhancing therapy.

### BACKGROUND

The following description includes information that may be useful in understanding the present invention. It is not an admission that any of the information provided herein is prior art or relevant to the presently claimed invention, or that any publication specifically or implicitly referenced is prior art.

Systemic delivery of tumor microenvironment modulators, such as cytokines (IL-2, IL-12, IL-15), chemokines, or antibodies (anti-PD1, anti-CTLA4, TGFbeta blocking Abs) can cause significant side effects due to having to infuse very large doses in order to achieve meaningful local effect, or significant pleiotropic activities, which can also be compounded by a sometimes very long persistence of the infused drug in vivo.

Delivering these modulators in a more targeted way can be achieved through the engineering of cells to either constitutively or inducibly secrete them and providing these cells to the tumor microenvironment either through tumor homing or local injection. However, constitutive expression (e.g., using armored CAR-T cells) may lead to prolonged exposure (as long as the cells persist). Termination of expression may be achieved by killing the secreting cells using an inducible suicide mechanism (e.g. iCasp9). However, such mechanisms do not kill 100% of the cells reliably, and they depend on the systemic administration of an inducer molecule. While transient induction of expression can be achieved, it requires complicated engineering of cells with often proprietary inducible promoters, as well as systemic administration of an inducer molecule. Furthermore, transient expression through loading cells with mRNA ensures that secretion is limited in time, however, the carrier cells will potentially persist and the timing of mRNA translation and protein secretion may be variable for different molecules, and therefore, not suited to all situations.

In order to ensure that an active molecule is not administered at harmful doses or that it is present in meaningful amount at the site(s) where it is needed, local injection of the product can be administered (e.g., intratumor injection, injection in resection space, intradermal). However, the targeted site may be difficult to access (e.g., brain tumors) or it may not be accessible for injection, not defined enough (diffuse tumors), or too multiple (metastases). One way to overcome the localized injection issue is to link the active molecule to a targeting agent, such as an antibody. Antibody-drug conjugates (ADC) are not able to access all parts of the body (such as the brain) and may still display dose-related toxicities. Another approach to localized injection is to make the targeting agent a cell capable of homing to the right site and capable of secreting the active molecule. Immune cells, for example, are able to reach tumor sites in all places of the body. Accordingly, engineering T-cells or NK cells to express the active molecule can be an efficient way of ensuring local delivery in the tumor. T-cells expressing IL-12 (either constitutively or inducibly) have been generated, as well as IL-15 expressing NK-cells. However, since these cells currently are autologous (i.e. from the patient), they can expand in vivo making it difficult to control the amount of active molecule and duration of exposure to the active molecule. While the introduction of suicide systems into engineered cells has been shown to successfully limit expansion in the body, such systems do not achieve 100% complete removal of the engineered cells. Moreover, engineering of autologous T- or NK-cells is labor-intensive and expensive, and limited to a single patient. Efforts to engineer allogeneic T- or NK-cells are ongoing but these cells have a high likelihood of being rejected by the patient's immune system.

In view of the above, there are many existing problems and disadvantages with the therapeutic use of engineered T cells and NK cells in a patient. Thus, there is still a need to improve the function of engineered T-cells or NK cells in order to effectively target these cells and their immune active molecule therapy to the tumor microenvironment while limiting overexposure of the therapy in the patient.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides a composition and methods in which a recombinant natural killer (NK) cell or T-cell is irradiated to thereby confer transient anti-tumor treatment to a subject and limiting adverse effects in the treatment of a tumor in the subject.

For effective targeting to a tumor microenvironment, the recombinant NK cell or T-cell to be irradiated is transfected with one or more recombinant nucleic acids. Preferably, the NK cell or T-cell to be irradiated is transfected with one or more recombinant nucleic acids encoding i) a homing receptor and/or cytokine, ii) an antigen binding protein (ABP) or chimeric antigen receptor (CAR) that specifically binds to a target antigen, iii) an Fc Receptor, and/or iv) a secreted immune modulator, wherein the one or more nucleic acids are operably linked to a promoter. In more preferred embodiments, the recombinant NK cell or T-cell is a NK-92 cell and the transfected nucleic acid is a tricistronic or quadricistronic vector with expression controlled by single promoter. Most preferably, the recombinant NK-92 cells to be irradiated are transfected with a quadricistronic vector encoding i) a cytokine, ii) an antigen binding protein (ABP) or chimeric antigen receptor (CAR) that specifically binds to a target antigen, iii) an Fc Receptor, and iv) a secreted immune modulator selected from a TGFβ inhibitor and/or IL-12.

In typical embodiments, the TGF-beta inhibitor is a TGF-beta trap. Additionally, the TGF-beta trap may comprise an extracellular domain of a TGFβRII molecule. More specifically, the TGF-beta trap comprises a single chain dimer of the TGF-beta Receptor II ectodomain (TGFbetaRIIecd).

Additionally or alternatively, the cytokine transfected in the recombinant NK cell or T-cell cell to be irradiated is IL2, IL-15, er-IL2, or er-IL15.

Additionally or alternatively with the above, the antigen binding protein transfected in the recombinant NK cell or T-cell to be irradiated specifically binds a tumor associated antigen. Typically, the tumor associated antigen is selected from CD19, CD20, GD2, HER-2, CD30, EGFR, FAP, CD33, CD123, PD-L1, IGFIR, CSPG4, or B7-H4.

Additionally or alternatively with the above, the antigen binding protein transfected in the the recombinant NK cell or T-cell to be irradiated binds an immune modulator protein in a tumor. Examples of immune modulator proteins in a tumor include CTLA-4, PD-1, IDO-1, CD39, or CD73.

Additionally or alternatively with the above, the Fc Receptor transfected in the recombinant NK cell or T-cell to be irradiated is CD16 or a high affinity CD16.

Additionally or alternatively with the above, the antigen binding protein transfected in the recombinant NK cell or T-cell to be irradiated comprises the chimeric antigen receptor (CAR).

Additionally or alternatively with the above, the promoter transfected in the recombinant NK cell or T-cell to be irradiated comprises at least one nuclear factor of activated T (NFAT) binding domain.

In some embodiments, the homing receptor is a G protein-coupled receptor (GPCR), a chemokine receptor, a cytokine receptor, a cell adhesion molecule, a selectin, or an integrin.

Aspects of the inventive subject matter also include a composition of the recombinant NK cell or T-cell irradiated as disclosed herein in combination with a pharmaceutically acceptable excipient.

Aspects of the inventive subject matter also include a method of treating a cancer or a tumor in a subject, the method including administering to the subject a therapeutically effective amount of the irradiated recombinant NK cell or T-cells and compositions as disclosed herein, wherein administration treats the cancer or reduces the size of the tumor in the subject. In typical embodiments, the irradiated recombinant NK cell or T-cells do not proliferate after exposure to irradiation and have transient activity. For example, the irradiated recombinant NK cell or T-cells transfected with one or more recombinant nucleic acids encoding i) a homing receptor and/or cytokine, ii) an antigen binding protein (ABP) or chimeric antigen receptor (CAR) that specifically binds to a target antigen, iii) an Fc Receptor, and/or iv) a secreted immune modulator confer anti-tumor activity for up to 72 hours, for example, up to 48 hours.

In preferred embodiments, methods of treating a cancer or a tumor in a subject include administering to the subject a therapeutically effective amount of the irradiated recombinant NK-92 cells as disclosed herein which are stably transfected with one or more recombinant nucleic acids encoding i) a homing receptor and/or a cytokine, ii) an antigen binding protein (ABP) or chimeric antigen receptor (CAR) that specifically binds to a target antigen, iii) an Fc Receptor, and/or iv) a secreted immune modulator, wherein the one or more nucleic acids are operably linked to a promoter. More preferably, the irradiated recombinant NK-92 cells are transfected with a quadricistronic vector encoding i) a cytokine, ii) an antigen binding protein (ABP) or chimeric antigen receptor (CAR) that specifically binds to a target antigen, iii) an Fc Receptor, and iv) a secreted TGFβ inhibitor and/or IL-12.

Similar aspects of the inventive subject matter also include a method of reducing cancer metastasis in a subject including administering a therapeutically effective amount of the irradiated modified NK-92 cells or compositions as disclosed herein to the subject, thereby reducing cancer metastasis in the subject.

In preferred embodiments, methods of administering of the irradiated modified NK-92 cells or compositions as disclosed herein wherein 1×10³ to 1×10¹⁰, per m² of the modified NK-92 cells are administered to the subject. Additionally, administration of the irradiated modified NK-92 cells and compositions include administration parenterally, intravenously, peritumorally, or by infusion.

In exemplary embodiments, a method of reducing cancer metastasis, tumor size, and/or decrease in cancer cells in a patient includes administering the irradiated recombinant NK-92 cell disclosed herein, wherein prior to irradiation, the NK-92 cell is transfected with a recombinant nucleic acid encoding a secreted immune modulator, a chemokine or a cytokine receptor, and/or an antigen binding protein or chimeric antigen receptor (CAR) that specifically binds to a target antigen, all of which are operably linked to a promoter. More specifically, the secreted immune modulator includes IL-12 and/or a TGF-beta inhibitor. Preferably the TGF-beta inhibitor is a TGF-beta trap. In preferred embodiments, the modified NK-92 cell to be irradiated is transfected with a quadricistronic vector encoding i) a cytokine comprising IL-2 or IL-15, ii) an antigen binding protein (ABP) or chimeric antigen receptor (CAR) that specifically binds to a target antigen, iii) an Fc Receptor, and iv) a secreted immune modulator selected from a TGFβ inhibitor and/or IL-12.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a schematic of a genetically modified natural killer (NK-92) cell with possible tumor-targeting elements as indicated.

FIG. 2A is a graph of the number of viable wildtype (WT) NK-92 cells at 0, 24, 48, 72, 96, and 120 hours after irradiation at 0, 2, 5, 10, and 30 Gy as indicated.

FIG. 2B is a graph of the percent cell viability of the wildtype (WT) NK-92 cells as in FIG. 2A at 0, 24, 48, 72, 96, and 120 hours after irradiation at 0, 2, 5, 10, and 30 Gy as indicated.

FIG. 3 is a graph showing the percentage of viable cells at the indicated time points (hours) up to approximately 144 hours (6 days) after irradiation with 10 Gy in aNK and haNK cells with non-irradiated aNK and haNK cells as a control.

FIG. 4A illustrates one embodiment of a quadricistronic vector encoding a TGFβ-trap armored PD-L1 CAR nucleic acids.

FIG. 4B is a graph of cell concentration of non-irradiated and irradiated t-haNK (NK-92) cells as indicated and further disclosed herein (transfected with the quadricistronic vector or FIG. 4A), with Day 0, Day 1, Day 2 cell counts for each indicated set of cells shown left to right.

FIG. 4C is a graph of the concentration (pg/mL) of TGFβRIIecd measured per million cells for the non-irradiated and irradiated t-hank (NK-92) cells of FIG. 4B.

FIG. 4D illustrates one embodiment of a quadricistronic vector encoding an IL-12 armored PD-L1 CAR nucleic acids.

FIG. 5A is a graph showing the number of PD-L1 t-haNK cells per volume (cells×10⁶/milliliter (mL)) at Day 1, 2, 3, 4, 7, 10, 15, 21, 28, and 35 after 0, 2.5, 5, 10, 15, 20, and 30 Gy irradiation as indicated.

FIG. 5B is a table of the raw data collected for the PD-L1 t-haNK cells as graphed in FIG. 5A.

FIG. 6 shows brightfield images captured using an EVOS microscope to visualize cell morphology of the PD-L1 t-haNK cells at Day 0, 2, 9, 21, 30, and 35 after 0, 2.5, 5, 10, 15, 20, or 30 Gy radiation, as indicated.

FIG. 7A shows cytometry images from an Annexin Assay of Annexin V stained PD-L1 t-haNK cells at Day 0, 2, 4, 17, 30, and 35 after 0, 2.5, 5, 10, 15, 20, or 30 Gy radiation, as indicated.

FIG. 7B shows cytometry images from a glutathione (GSH) Vitality Assay of PD-L1 t-haNK cells at Day 0, 2, 4, 17, 30, and 35 after 0, 2.5, 5, 10, 15, 20, or 30 Gy radiation, as indicated.

### DETAILED DESCRIPTION

The inventors have surprisingly discovered that irradiation of recombinant (i.e., engineered or modified allogeneic) T- or NK-cells as disclosed herein can maintain therapeutic effectiveness of these immune therapy delivery cells for treatment of cancer tumors in a patient while also limiting overexposure and side effects in the patient. In particular, the inventors advantageously discovered that irradiated NK cells engineered to express a secreted immune modulator are effectively inhibited from replication (e.g., cell proliferation) while maintaining transient activity of the irradiated recombinant NK cell including transient activity of the secreted immune modulator(s). One should appreciate that limiting patient overexposure and/or side effects of an allogeneic cellular therapy is indeed desirable for engineered tumor-targeted cells.

Considering allogeneic cells such as a recombinant NK cell can induce harmful effects when infused in a subject (e.g., a cancer patient), it was surprisingly discovered that irradiating allogeneic cells transfected with a nucleic acid encoding a secreted immune modulator (e.g., a TGFβ inhibitor or IL-12) inhibits cell proliferation as expected, while maintaining activity and of the secreted immune modulator for a transient period after irradiation. Unlike cell surface expressed recombinant molecules, secreted cytokines are not membrane-bound and are dependent on the host cells nuclear machinery for expression. Gamma (Y)-irradiation destroys the host cell nuclear machinery, thereby arresting cell proliferation. Additionally, γ-irradiation inhibits expression of genes in pathways related to cell-type specific immunity, and in particular, NK cells. See, Paul et al., 2013, Radiat. Res., 180:575-583. Remarkably, however, γ-irradiated NK cells are capable of expressing a secreted cytokine (e.g., a TGFβ inhibitor or IL-12). Accordingly, γ-irradiation of recombinant NK cells transfected with a quadricistronic vector encoding i) a homing receptor and/or a cytokine, ii) an antigen binding protein (ABP) or chimeric antigen receptor (CAR) that specifically binds to a target antigen, iii) an Fc Receptor, and iv) a secreted immune modulator inhibited the recombinant NK cells from replication while maintaining transient activity of the encoded recombinant molecules including the secreted immune modulators. For example, transient activity was maintained for up to 72 hours, including, for example, up to 48 hours.

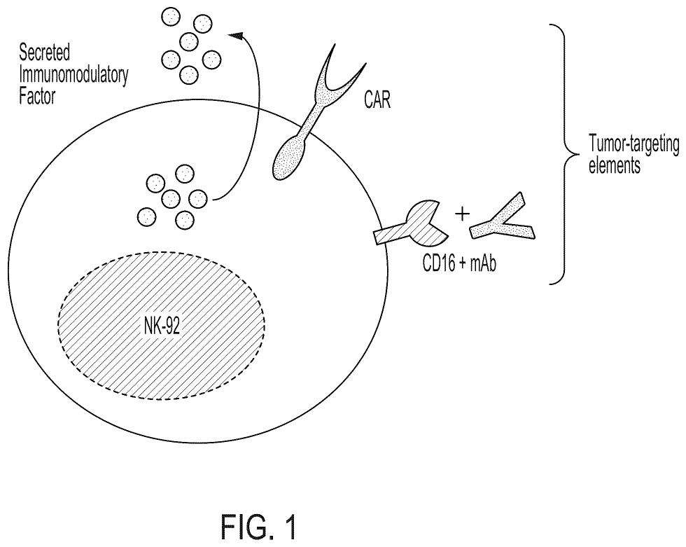

Accordingly, the contemplated inventive subject matter includes the rendering of and treatment with irradiated tumor-targeted cells (e.g., T-cells or NK-cells) engineered to constitutively express secreted modulators, antigen binding proteins, an Fc Receptor, and a homing receptor or cytokine/chemokine to aide in targeting the tumor. The schematic in FIG. 1 depicts an exemplary tumor targeted cell, the NK-92 cell, having the tumor targeting elements of a chimeric antigen receptor (CAR) and CD16 receptor for targeting a tumor along with secreted immune-modulatory molecules (e.g., TGFβ inhibitor and/or IL-12). Examples of these engineered NK-92 cells capable of homing to a tumor are disclosed, for example, in WO2020/028656, the entire contents of which are herein incorporated by reference. As disclosed herein in more detail, controlled irradiation of allogeneic engineered T- or NK-cells maintains effective therapeutic function of these tumor-targeted cells, while limiting the proliferation of these cells for about 48 to 72 hours (e.g., 2 to 3 days).

Preferably, the engineered NK cells include a cytotoxic activated Natural Killer cell line (e.g., NK-92) as the basis to improve immunotherapies to cancer cells and tumors, and/or to increase homing (migration) towards a target of interest. In some embodiments, the NK-92 cells are engineered to express a homing receptor known to direct lymphocytes to lymph nodes when expressed. In some embodiments, the NK cells are transfected with a nucleic acid encoding a secreted cytokine that modulates the tumor microenvironment or an inhibitor that blocks a cytokine that modulates the tumor microenvironment.

More preferably, prior to irradiation, the NK cells are transfected with a nucleic acid to express a secreted immune modulator (e.g., a TGFβ inhibitor and/or IL-12), and an antigen binding protein (ABP) or chimeric antigen receptor (CAR) that specifically binds to a target antigen (e.g., an anti-PD-L1), and optionally one or both selected from a homing receptor and an Fc Receptor, wherein the nucleic acid is operably linked to a promoter in a tricistronic construct (vector). In an additional preferred embodiment, the NK cells are transfected with a nucleic acid to express a secreted immune modulator (e.g., a TGFβ inhibitor and/or IL-12), an antigen binding protein (ABP) or chimeric antigen receptor (CAR) that specifically binds to a target antigen (e.g., an anti-PD-L1), and an Fc Receptor (e.g., CD16 or CD16-158V), wherein the nucleic acid is operably linked to a promoter in a tricistronic construct (vector). In another preferred embodiment, the NK cells are transfected with a nucleic acid to express a secreted immune modulator (e.g., a TGFβ inhibitor and/or IL-12), an antigen binding protein (ABP) or chimeric antigen receptor (CAR) that specifically binds to a target antigen (e.g., an anti-PD-L1), and a homing receptor, wherein the nucleic acid is operably linked to a promoter in a tricistronic vector.

Most preferably, the NK cells are transfected with a nucleic acid to express a secreted immune modulator (e.g., a TGFβ inhibitor and/or IL-12), a homing receptor, an Fc Receptor (e.g., CD16 or CD16-158V), and an antigen binding protein (ABP) or chimeric antigen receptor (CAR) that specifically binds to a target antigen, wherein the nucleic acid is operably linked to a promoter in a quadricistronic vector.

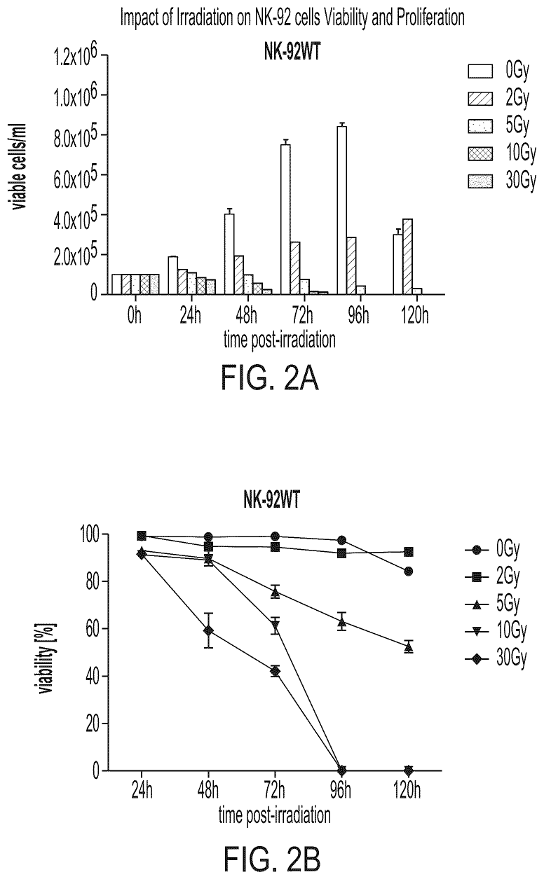

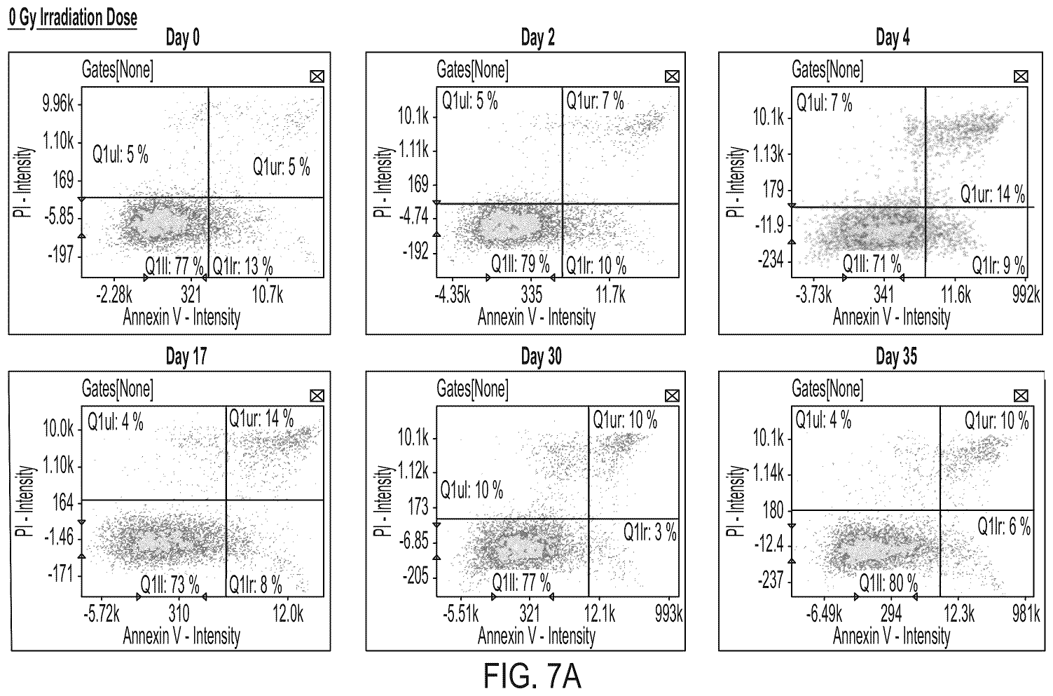

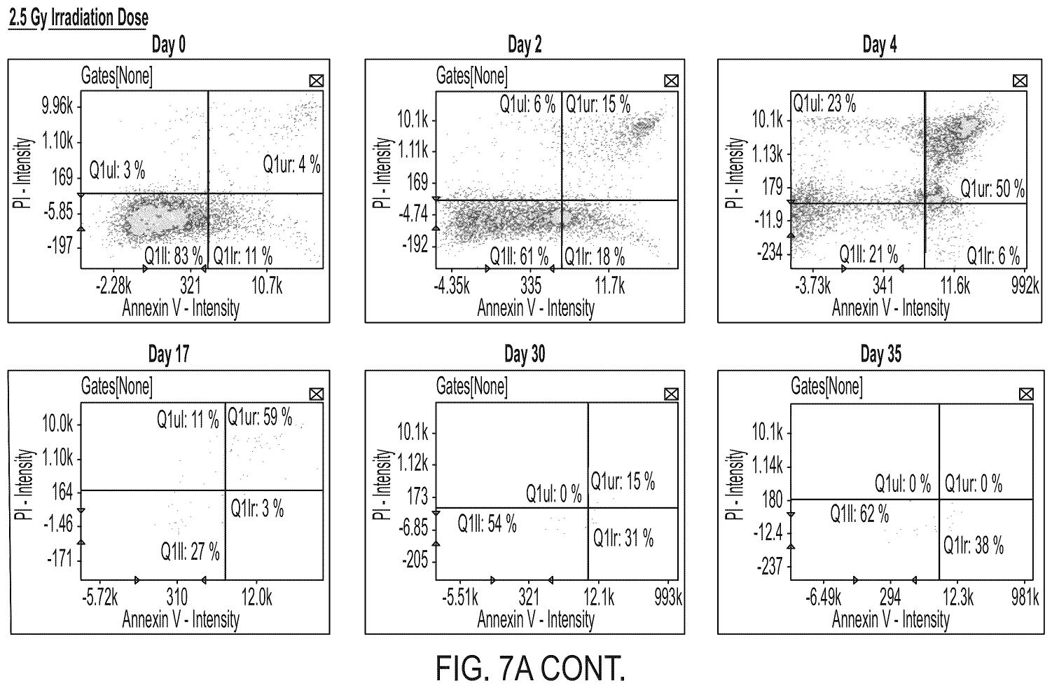

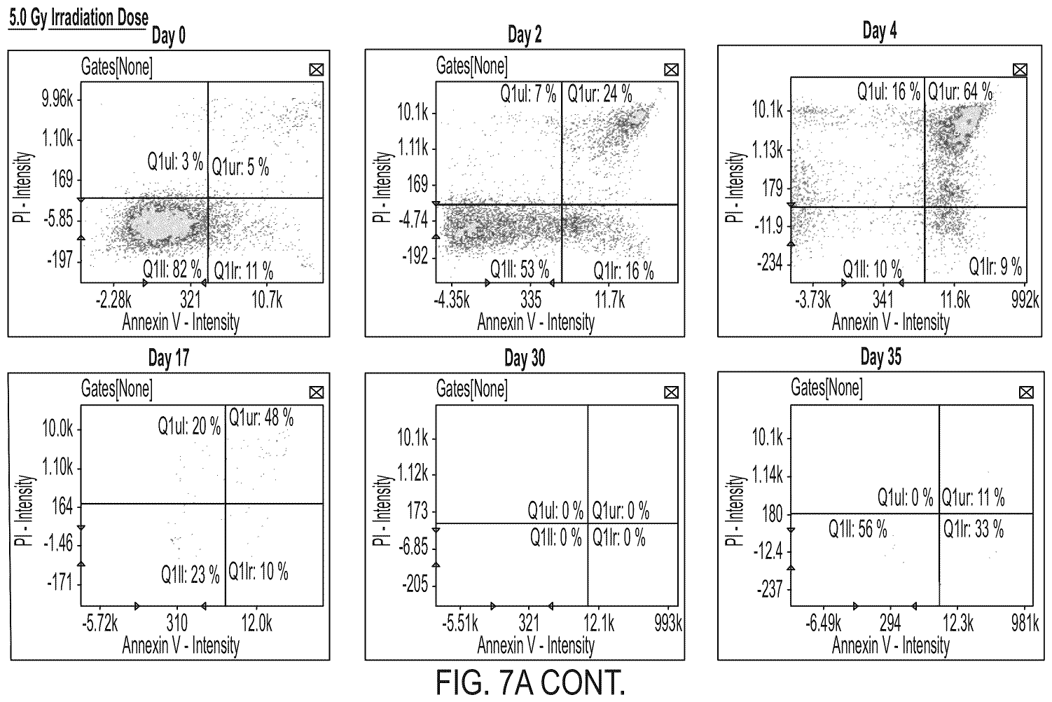

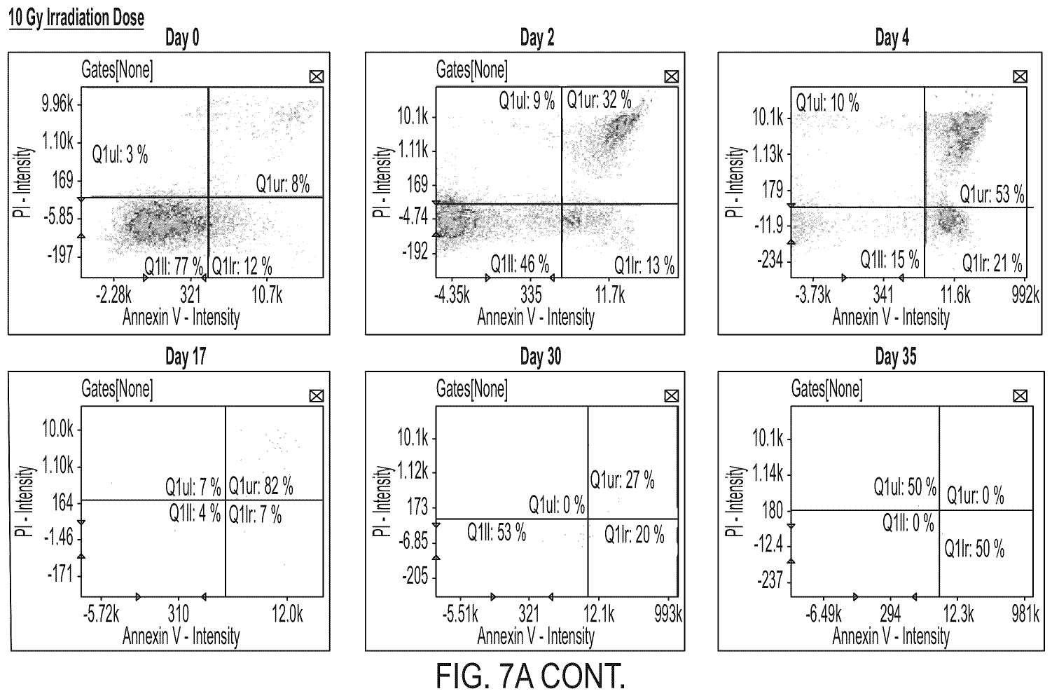

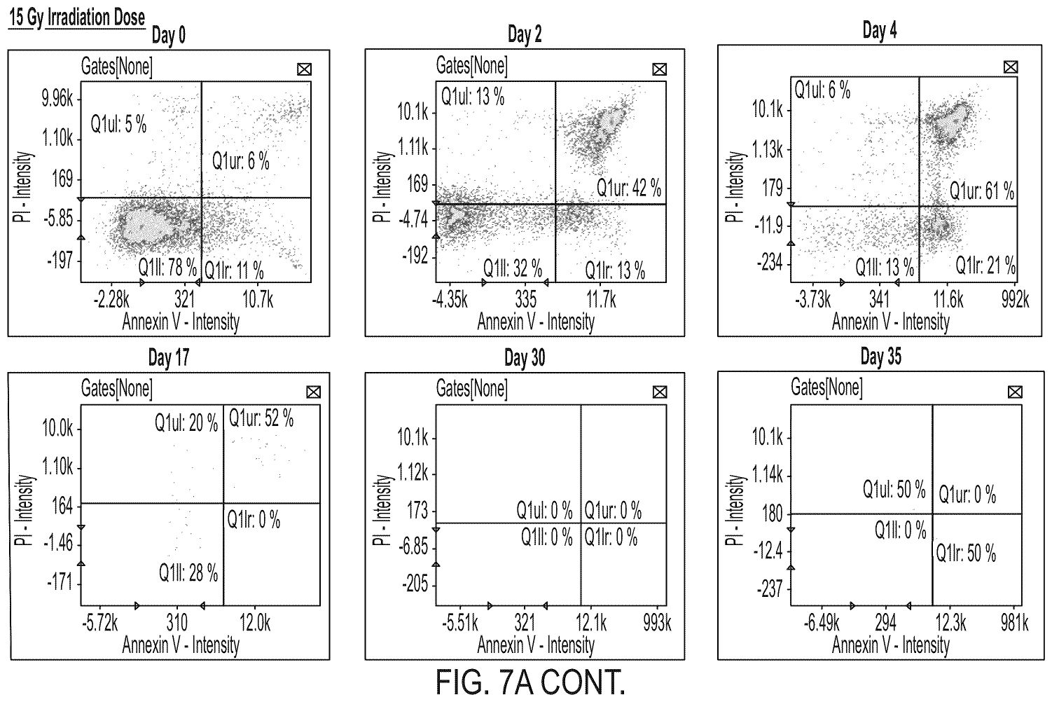

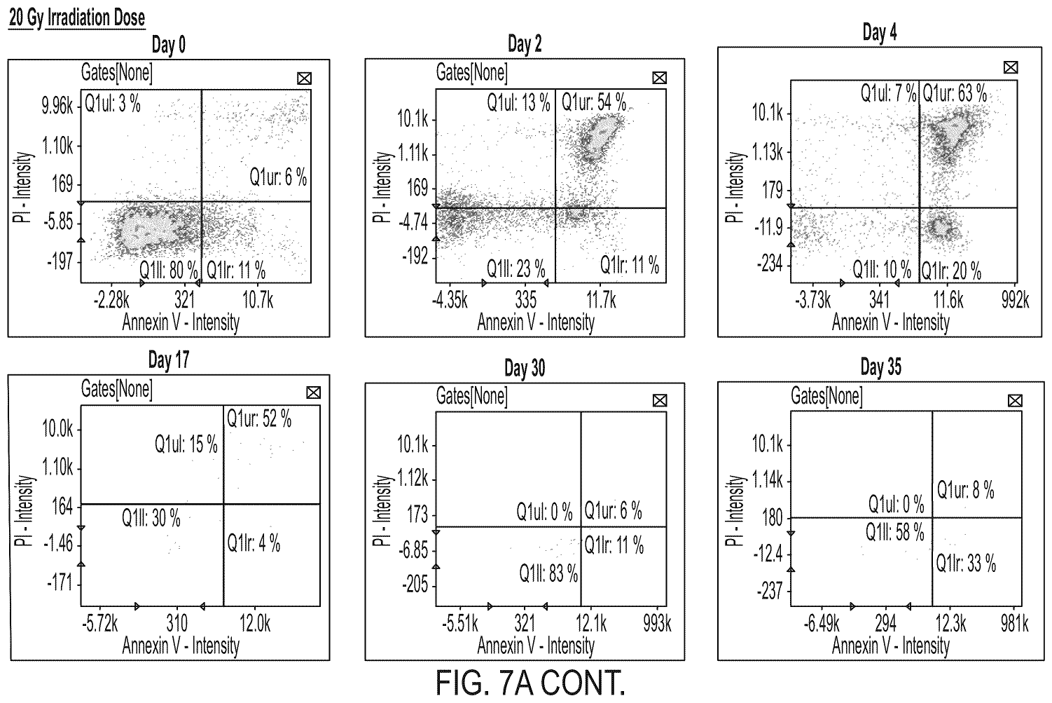

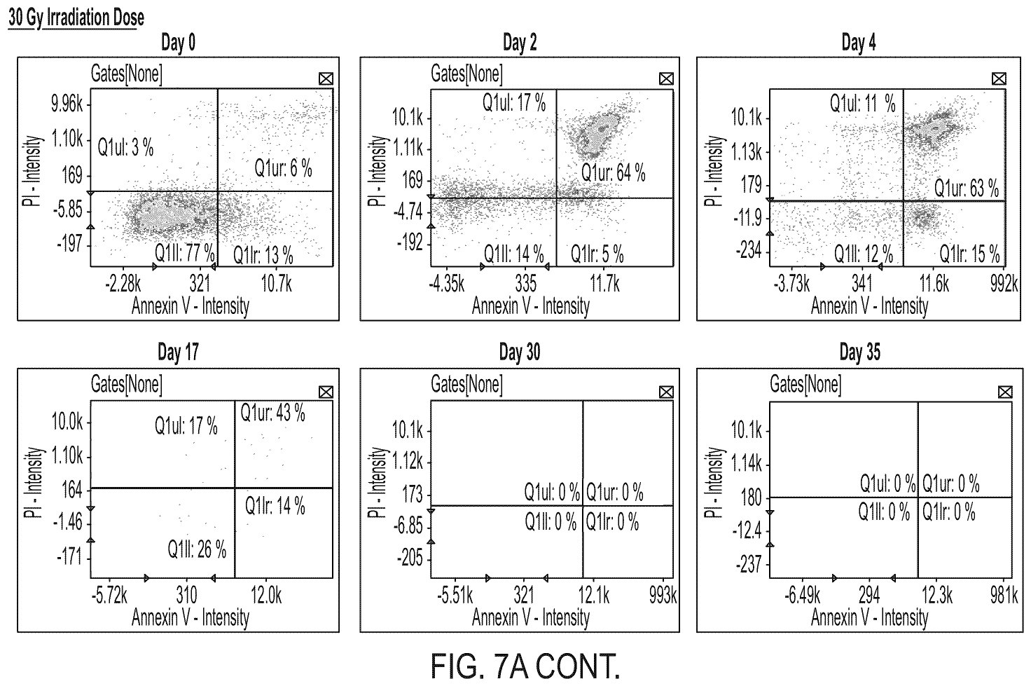

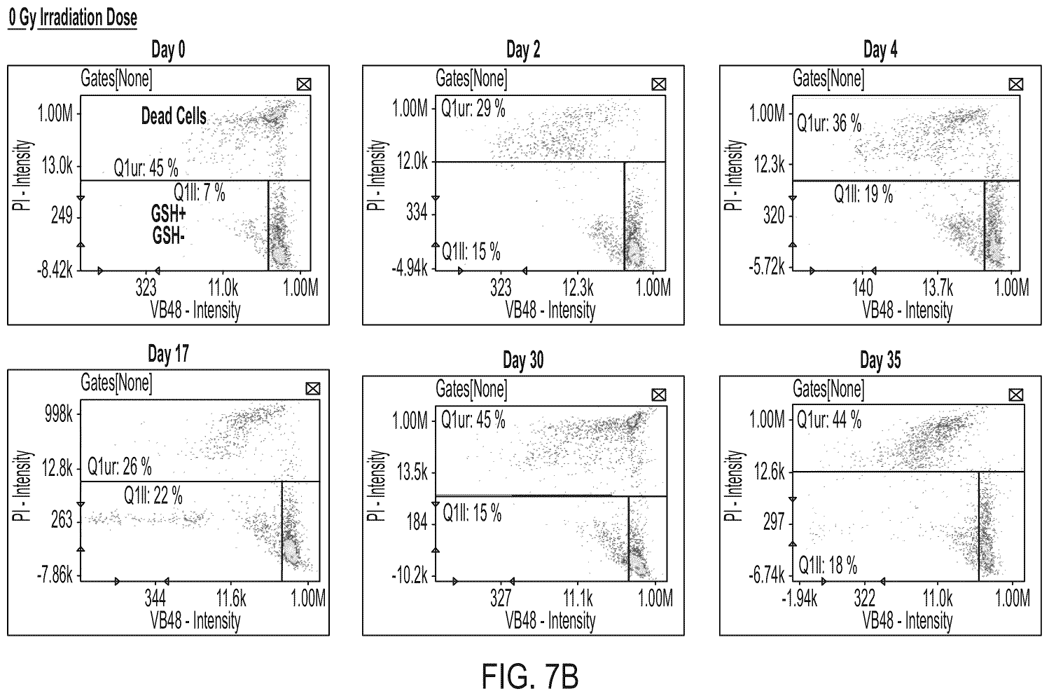

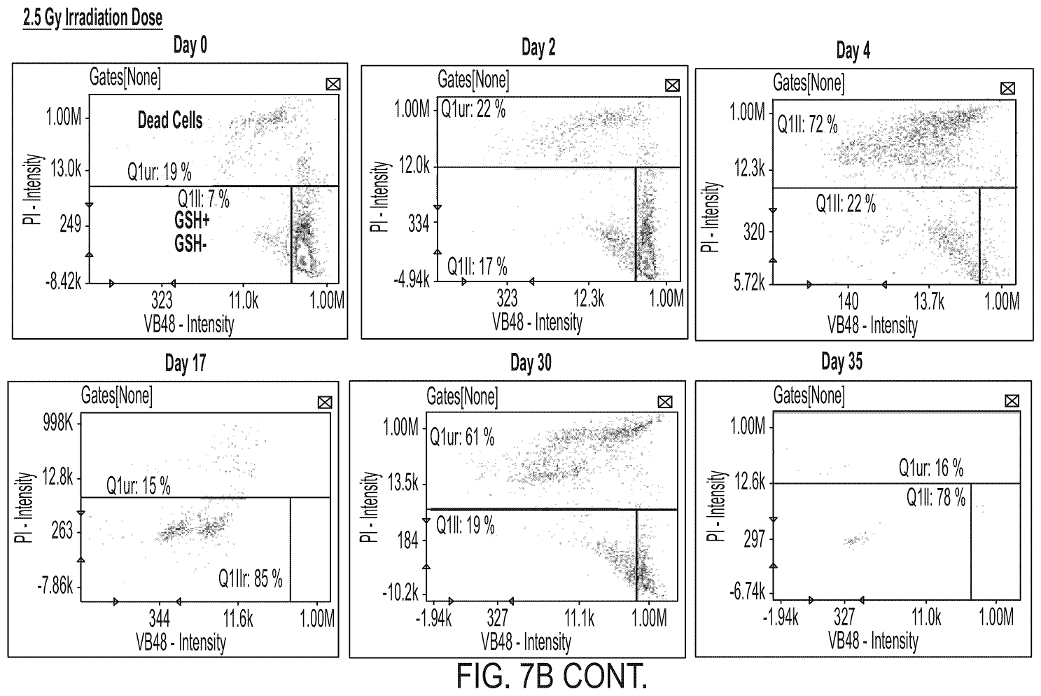

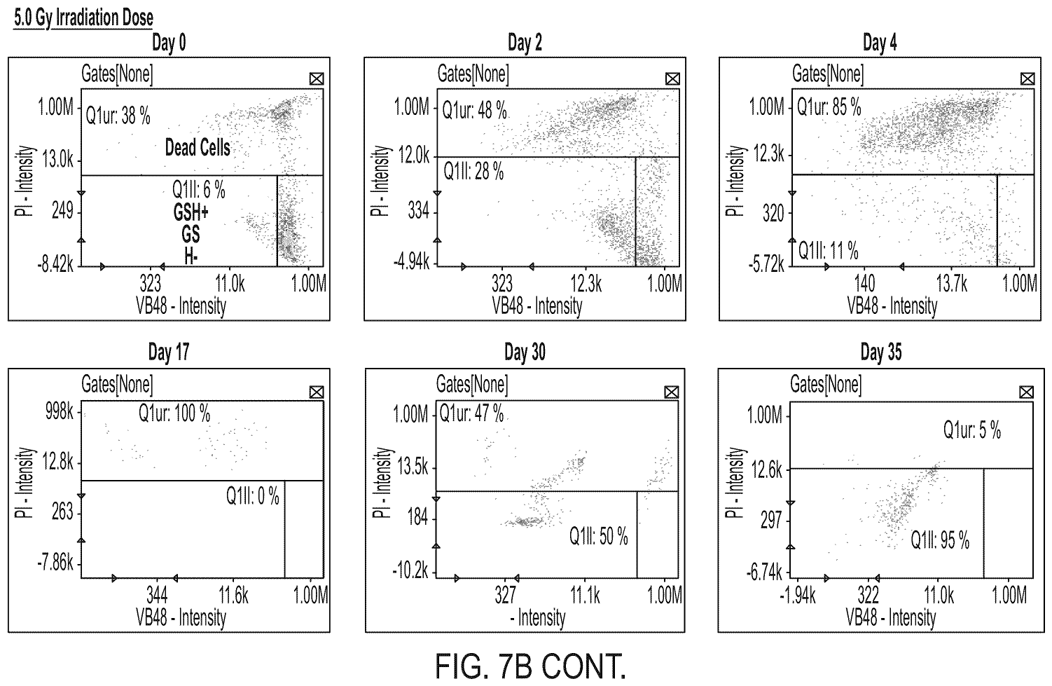

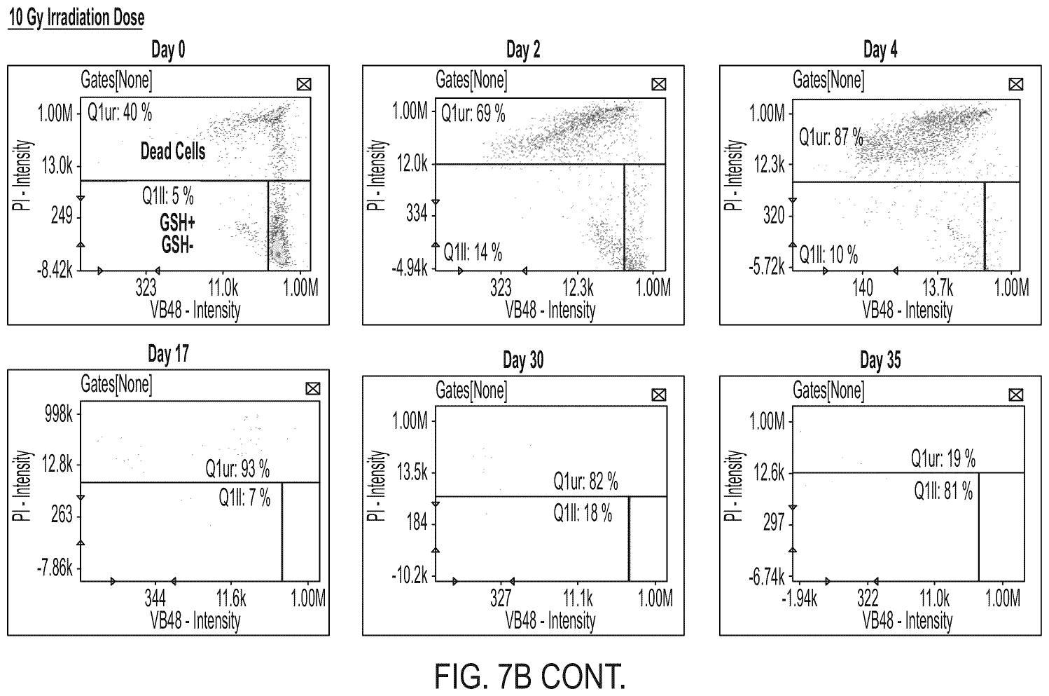

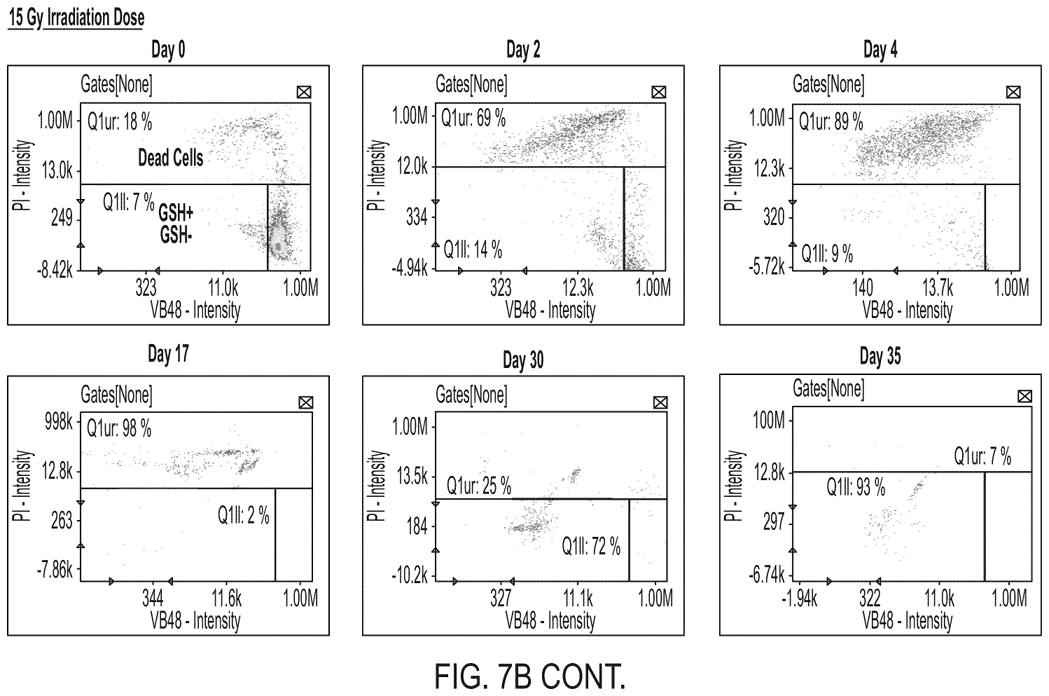

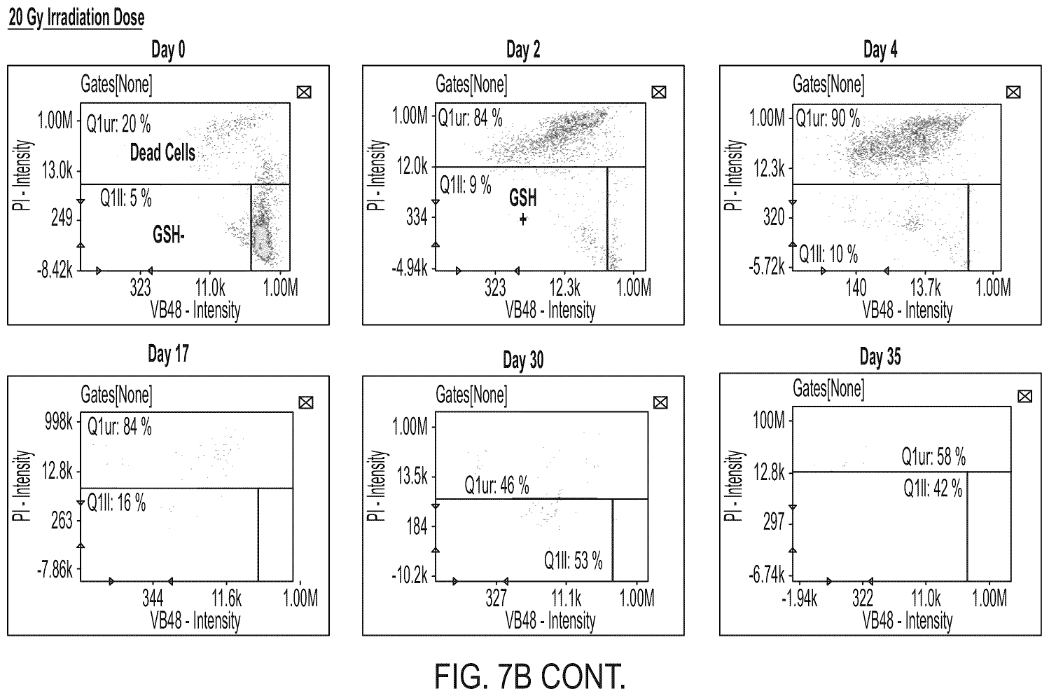

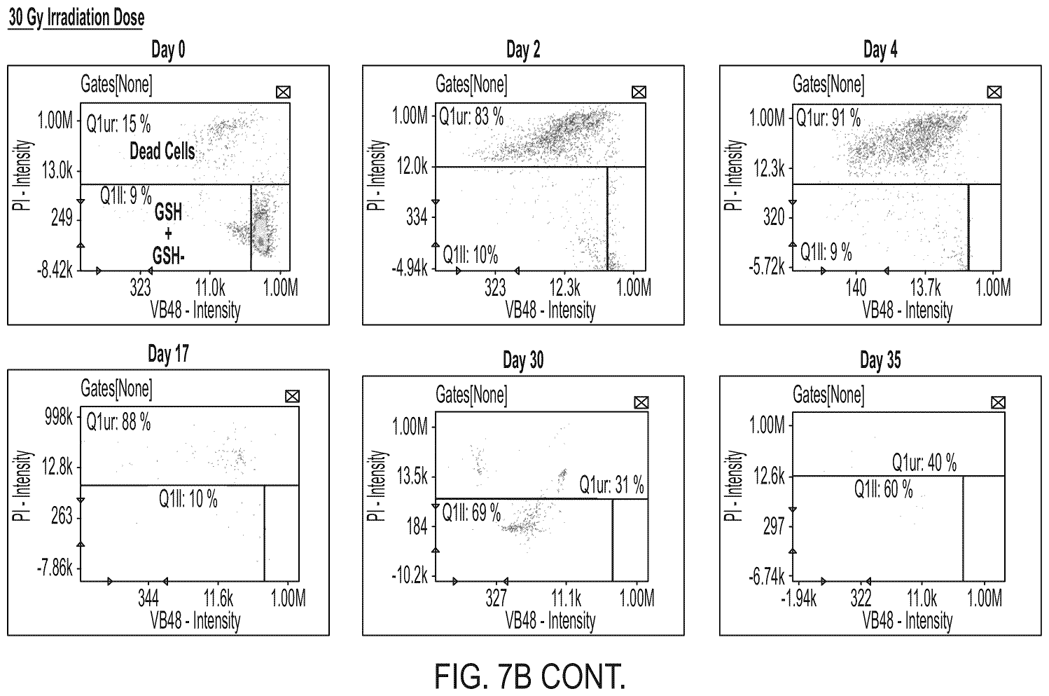

As disclosed herein, therapeutic function may be determined by assaying the integrity of the cell and the recombinant molecules transfected therein as set forth in FIGS. 2A, 2B, 3, 4A, 4B, 4C, 5A, 5B, 6, 7A, and 7B. Cell integrity may be measured by assaying the presence and/or function of known cellular molecules including recombinant as well as endogenous molecules. For example, with reference to FIG. 7B, the vitality of the cell after irradiation is measured by the presence of intracellular reduced thiols (glutathione; GSH).

Specifically, the allogeneic modified T- or NK-cells may be irradiated with of between 5 to 30 gray (Gy). As understood by persons skilled in the art, the gray is a derived unit of ionizing radiation dose in the International System of Units. The gray (Gy) is defined as the absorption of one joule of radiation energy per kilogram of matter.

To maintain the function of expression of tumor targeting elements while limiting cellular proliferation, engineered allogeneic T- or NK-cells may be irradiated with 5, 6, 7, 8, 9, 10, 11, 12, 13, 14, 15, 16, 17, 18, 19, 20, 21, 22, 23, 24, 25, 26, 27, 28, 29, or 30 Gy. As can be determined by the skilled person, some engineered T- or NK-cells may require irradiation in the range of between 5 to 15 Gy (e.g., 5, 6, 7, 8, 9, 10, 11, 12, 13, 14, or 15 Gy) to maintain therapeutic effectiveness while limiting proliferation of the cells to no more than 72 hours. In still other embodiments, the range of irradiation may be of between 5 to 10 Gy (e.g., 5, 6, 7, 8, 9, or 10 Gy).

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

As used herein, the term “transfect” refers to the insertion of nucleic acid into a cell. Transfection may be performed using any means that allows the nucleic acid to enter the cell. DNA and/or mRNA may be transfected into a cell. Preferably, a transfected cell expresses the gene product (i.e., protein) encoded by the nucleic acid.

The term “homing receptor” refers to a receptor that activates a cellular pathway that results directly or indirectly in the cell migrating toward a target cell or tissue. For example, homing receptors expressed by leukocytes are used by leukocytes and lymphocytes to enter secondary lymphoid tissues via high endothelial venules. Homing receptors can also be used by cells to migrate toward the source of a chemical gradient, such as a chemokine gradient.

Non-limiting examples of homing receptors include G protein-coupled receptors such as chemokine receptors, including but not limited to CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, or the receptor for CXCL14; cytokine receptors; cell adhesion molecules such as selectins, including L-selectin (CD62L); integrins such as alpha4beta7 integrin, LPAM-1, and LFA-1. Homing receptors generally bind to cognate ligands on the target tissues or cell. In some embodiments, homing receptors bind to Addressins on the endothelium of venules, such as mucosal vascular addressin cell adhesion molecule 1 (MAdCAM-1). In some embodiments, the nucleic acid encoding CCR7 has at least 90%, 91%, 92%, 93%, 94%, 95%, 96%, 97%, 98% or 99% identity to SEQ ID NO:1. In some exemplary embodiments, the chemokines and homing receptors contemplated herein may comprise a polypeptide sequence or a polynucleotide sequence having at least 80%, 85%, 90%, 91%, 92%, 93%, 94%, 95%, 96%, 97%, 98% or 99% identity to SEQ ID NO: 44 (CCR7 a.a. sequence), or SEQ ID NO: 45 (CCL19 a.a. sequence), or SEQ ID NO: 46 (CCL21 a.a. sequence), or SEQ ID NO: 47 (CXCR2 n.t. sequence), or SEQ ID NO: 48 (CXCR2 a.a. sequence), or SEQ ID NO: 49 (CXCL14 n.t. sequence), or SEQ ID NO: 50 (CXCL14 a.a. sequence), or SEQ ID NO: 51 (CD62L n.t. sequence), or SEQ ID NO: 52 (CD62L a.a. sequence), or SEQ ID NO: 53 (IL-8 n.t. sequence), or SEQ ID NO: 54 (IL-8 a.a. sequence), or SEQ ID NO: 55 (CXCL1 n.t. sequence), or SEQ ID NO: 56 (CXCL1 a.a. sequence).

As used herein, “immunotherapy” refers to the use of NK-92 cells, modified or unmodified, naturally occurring or modified NK cell or T-cell, whether alone or in combination, and which are capable of inducing cytotoxicity when contacting a target cell.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

As used herein, “natural killer (NK) cells” are cells of the immune system that kill target cells in the absence of a specific antigenic stimulus, and without restriction according to major histocompatibility complex (MHC) class. NK cells are characterized by the presence of CD56 and the absence of CD3 surface markers.

The contemplated subject matter includes irradiating recombinant allogeneic NK cells or T-cells with 5 to 30 Gy. Preferably, the recombinant allogeneic cells are NK-92 cells irradiated with 5 to 30 Gy. With reference to FIGS. 2A-2B, cell viability of wild type NK-92 cells was measured after irradiation as indicated. As disclosed and referenced herein, NK-92 cells may be engineered to create unique cell lines that can be grown in culture indefinitely. In clinical trials, they have been shown to be well tolerated by patients and to not induce harmful immune responses. They are available as an “off-the-shelf” product without patient restriction, and therefore more advantageous to use than autologous T- or NK-cells. They can be made to preferentially home to tumor sites, thereby enabling local delivery of secreted molecules. Advantageously, the recombinant NK cells disclosed herein maintain homing, cytotoxic, and secretory functions for 48 to 72 hours after exposure with lethal doses of gamma radiation, but subsequently die, thereby eliminating issues associated with cellular expansion and extended persistence. The recombinant NK cells as disclosed herein can be irradiated and infused repeatedly, which allows control over duration of exposure to the active molecules.

In exemplary embodiments, NK-92 cells to be irradiated may be genetically engineered by transfection or transduction with DNA constructs encoding one or multiple genes. In particular, the inventive subject matter comprises modified NK-92 cells to be irradiated that are capable of modulating the tumor microenvironment. These modified NK-92 cells preferably comprise a multi-cistronic vector comprising one or more nucleic acids encoding i) a homing receptor and/or cytokine, ii) an antigen binding protein (ABP) or chimeric antigen receptor (CAR) that specifically binds to a target antigen, iii) an Fc Receptor (e.g., CD16 or CD16-158V), and/or iv) a secreted immune modulator, wherein the one or more nucleic acids are operably linked to a promoter.

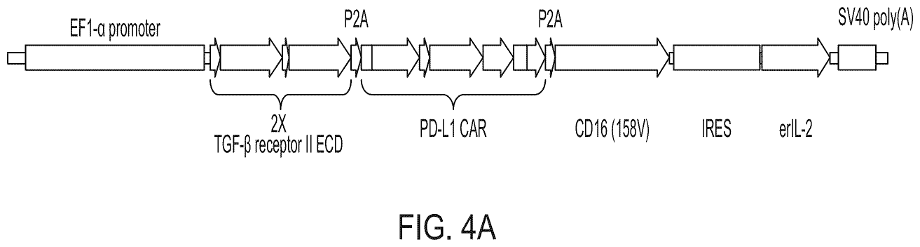

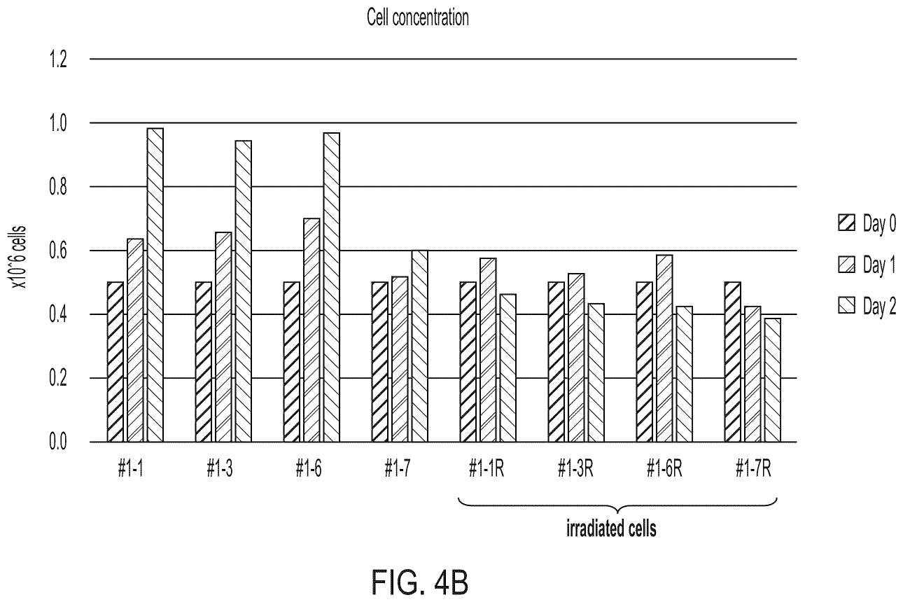

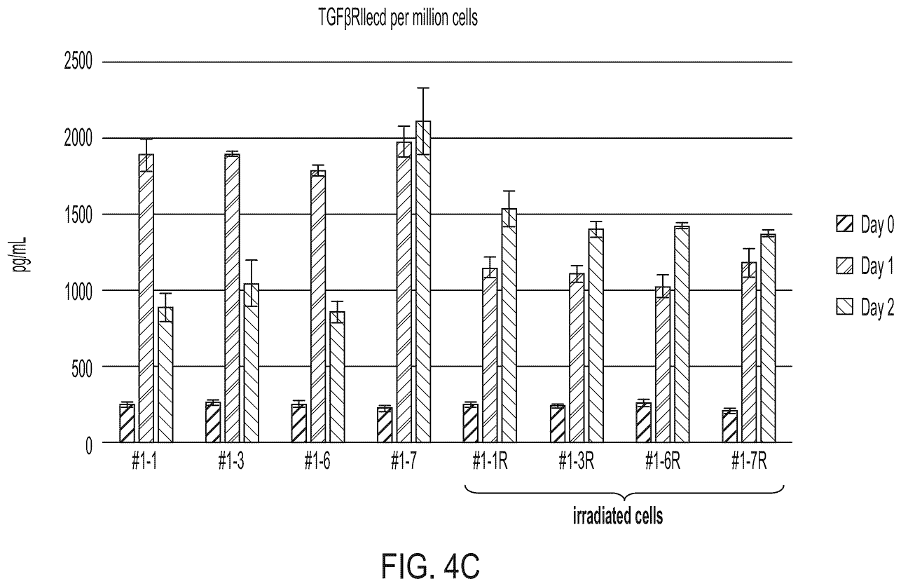

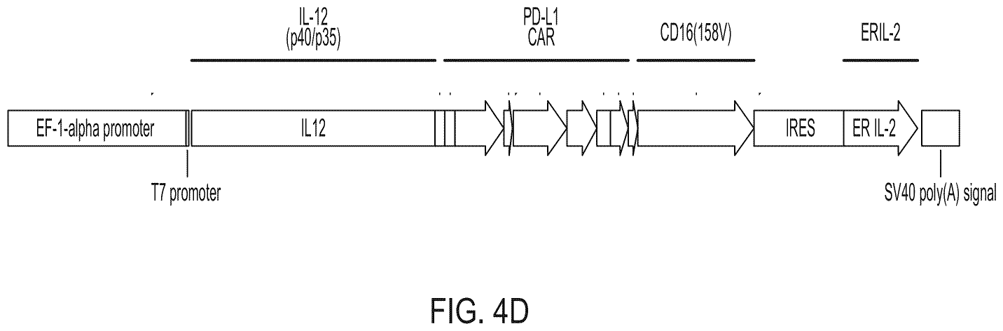

Notably, NK-92 cells engineered to constitutively express a secreted TGFbeta-trap molecule or a single chain IL-12 have been found to be able to secrete high quantities of active molecules (in the ng/ml range). Moreover, it has been observed that NK-92 cells have the ability to home to tumor sites, and that homing can be enhanced by expression of a tumor-specific CAR molecule (such as PD-L1 CAR) and/or a chemokine receptor. For example, NK-92 cells engineered to express the CCR7 chemokine receptor have also been shown to home to CCL19-producing tumors more efficiently than parental NK-92 cells. NK-92 cells irradiated with a clinically compatible dose of radiation (e.g., up to 10, 15, 20, or 30 Gy) survive up to 48 hours or up to 72 hours, while being able to maintain cytotoxic and secretory functions (FIG. 4B-4C). Infusion of irradiated engineered NK-92 cells therefore provides a way to control delivery of secreted molecules in space (tumor-homing) as well as in time (because of the limited lifespan of irradiated cells). In some embodiments, recombinant NK-92 cells as disclosed herein that have been irradiated with a clinically compatible dose of 10 or 15 Gy gamma (Y) irradiation survive up to 24, 30, 36, 42, 48, 54, 60, 66, or 72 hours while being able to maintain cytotoxic and secretory functions.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due in part to cytolysis.

The term “kill” with respect to a cell/cell population is directed to include any type of manipulation that will lead to the death of that cell/cell population.

The term “NK-92” refers to natural killer cells derived from the highly potent unique cell line described in Gong et al. (1994), rights to which are owned by NantKwest® (hereafter, “NK-92 cells”). The immortal NK cell line was originally obtained from a patient having non-Hodgkin's lymphoma. Unless indicated otherwise, the term “NK-92” is intended to refer to the original NK-92 cell lines as well as NK-92 cell lines that have been modified (e.g., by introduction of exogenous genes). NK-92 cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92, NK-92-CD16, NK-92-CD16-γ, NK-92-CD16-ξ, NK-92-CD16(F176V), NK-92MI, and NK-92-CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK® cells”). The term “haNK” refers to natural killer cells derived from the highly potent unique cell line described in Gong et al. (1994), rights to which are owned by NantKwest, modified to express CD16 on the cell surface (hereafter, “CD16+NK-92 cells” or “haNK cells”). In some embodiments, the CD16+NK-92 cells comprise a high affinity CD16 receptor on the cell surface. An example of a high affinity CD16 includes CD16-F158V. The term “taNK” refers to natural killer cells derived from the highly potent unique cell line described in Gong et al. (1994), rights to which are owned by NantKwest, modified to express a chimeric antigen receptor (hereafter, “CAR-modified NK-92 cells” or “taNK® cells”). The term “t-haNK” refers to natural killer cells derived from the highly potent unique cell line described in Gong et al. (1994), rights to which are owned by NantWest, modified to express CD16 on the cell surface and to express a chimeric antigen receptor (hereafter, “CAR-modified CD16+NK-92 cells” or “t-haNK cells”). In some embodiments, the t-haNK cells express a high affinity CD16 receptor on the cell surface.

In addition, irradiated recombinant NK-92 cells as disclosed herein may also include a promoter having NFAT binding domains (sequence) introduced into the promoter for expression of a cytokine, chemokine, and/or antigen binding protein. NK-92 cells engineered to express a luciferase reporter gene under the control of a nuclear factor of activated T cells (NFAT) transcription factor promoter sequence have been shown to induce high luciferase expression in response to stimulation on activating receptors that signal through the NFAT pathway (such as receptors that recruit CD3ξ or FceRIγ adaptor molecules). Accordingly, this inducible expression of a secreted molecule is dependent on the cells being activated by a suitable target and does not depend on an external inducer molecule. In one embodiment, the CD35 signaling domain contemplated herein may comprise a polypeptide sequence having at least 80%, 85%, 90%, 91%, 92%, 93%, 94%, 95%, 96%, 97%, 98% or 99% identity to SEQ ID NO: 40.

As disclosed in WO2020/028656 (the entire contents of which are herein incorporated by reference) target engagement of susceptible cell lines is shown to be recognized in NK-92 cells by activation of the NFAT transcription factor and its nuclear translocation. Target binding involving the FceRIγ or CD3zeta pathway (including ADCC or CAR mediated target recognition) is sufficient to induce NFAT activation in NK-92 cells. This was demonstrated by inserting a reporter cassette containing 3 NFAT response elements and a minimal promoter driving firefly luciferase. NFAT activation by the CD3zeta pathway through electroporation of CD19 CAR mRNA into this reporter cell line, followed by co-culture with SUP-B15 (CD19+, but resistant to non-specific cytotoxicity) resulted in luciferase expression.

In exemplary embodiments, the complete sequence for the NFAT response cassette driving CCL21+Poly-A, and a FRT-embedded blasticidin resistance gene driven by CMV, comprises a polynucleotide sequence having at least 80%, 85%, 90%, 91%, 92%, 93%, 94%, 95%, 96%, 97%, 98% or 99% identity to SEQ ID NO:8.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII-A (also called CD16) is a low affinity Fc receptor that binds to IgG antibodies and activates ADCC. FCγRIII-A are typically found on NK cells. NK-92 cells do not express FCγRIII-A. Fc-epsilon receptors (FcER) bind to the Fc region of IgE antibodies. In some embodiments, the CD16 receptor comprises a phenylalanine (F) to valine (V) substitution at amino acid position 158 (F158V) of the mature form of the polypeptide (SEQ ID NO: 12) (corresponding to position 176 of the full length form of the polypeptide comprising the signal sequence). In one embodiment, the Fc receptor comprises the nucleic acid sequence of SEQ ID NO: 13 or the amino acid sequence of SEQ ID NO: 12.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. For example, a CAR-expressing NK-92 cell as disclosed herein to be irradiated (e.g., and expressed together with a homing receptor, an Fc receptor, and a secreted cytokine) is targeted to cells expressing certain antigens on the cell surface, based on the specificity of the scFv domain. The scFv domain can be engineered to recognize any antigen, including tumor-specific antigens. For example, CD19-CAR recognizes CD19, a cell surface marker expressed by some cancers.

In additional embodiments, the recombinant CAR comprises a cytoplasmic domain of FcεRIγ. For example, the cytoplasmic domain of FceRIγ may comprise an amino acid sequence having at least 95% sequence identity to SEQ ID NO: 31. Alternatively, the cytoplasmic domain of FcεRIγ is encoded by a nucleic acid having at least 95% sequence identity to SEQ ID NO:32. In other embodiments, the CAR comprises a hinge region from CD8. In some embodiments, the CAR comprises a transmembrane domain from CD28.

Accordingly, the recombinant NK-29 cells may be transfected with a nucleic acid construct (e.g., quadricistronic vector) that comprises SEQ ID NO:31 (FcεRIγ intracellular cytoplasmic domain), SEQ ID NO:32 (FcεRIγ intracellular signaling domain minus transmembrane domain), SEQ ID NO: 33 (CD8 hinge region), SEQ ID NO: 34 (CD8 hinge region DNA), SEQ ID NO:35 (CD28 transmembrane domain) and/or SEQ ID NO:36 (CD28 transmembrane domain, minus ITAM or intracellular sequence). In one embodiment, the CD8 hinge region, CD28 transmembrane, and FcεRIγ (FceRIgamma) signaling domain amino acid sequence comprises a polypeptide or a polynucleotide sequence having at least 80%, 85%, 90%, 91%, 92%, 93%, 94%, 95%, 96%, 97%, 98% or 99% identity to SEQ ID NO: 37 or SEQ ID NO: 38. In one embodiment, the CAR scFv may comprise a polypeptide sequence having at least 80%, 85%, 90%, 91%, 92%, 93%, 94%, 95%, 96%, 97%, 98% or 99% identity to SEQ ID NO: 39.

Notably, the inventive subject matter includes irradiated modified NK-92 cells that are capable of transiently modulating the tumor microenvironment. The irradiated modified NK-92 cell preferably includes a quadricistronic vector comprising one or more nucleic acids encoding i) IL-12 or a TGF-beta trap, ii) an Antigen Binding Protein (ABP) or Chimeric Antigen Recpetor (CAR) that specifically binds to a target antigen, iii) an Fc Receptor such as CD16 or CD16-158V, and/or iv) a cytokine (e.g., erIL-2 or erIL-15), wherein the nucleic acid sequence is operably linked to a promoter.

In another aspect, the secreted cytokine that modulates the tumor microenvironment may be IL-12 or a TGF-beta inhibitor. Accordingly, the nucleic acid construct may encode for IL-12 and/or a TGF-beta inhibitor.

In exemplary embodiments, the IL-12 as contemplated herein may comprise a nucleic acid sequence with at least 80%, 85%, 90%, 91%, 92%, 93%, 94%, 95%, 96%, 97%, 98% or 99% identity to SEQ ID NO: 57 (p35 n.t. sequence), or SEQ ID NO: 59 (p40 n.t. sequence). The IL-12 contemplated herein may also comprise an amino acid sequence with at least 80%, 85%, 90%, 91%, 92%, 93%, 94%, 95%, 96%, 97%, 98% or 99% identity to SEQ ID NO: 58 (p35 a.a. sequence, isoform 1 precursor), or SEQ ID NO: 60 (p40 a.a. sequence, precursor).

In one exemplary embodiment, the IL-12 single chain p40_p35 sequence in IL-2/PD-L1 Quadricistronic vector may include a polypeptide sequence with at least 80%, 85%, 90%, 91%, 92%, 93%, 94%, 95%, 96%, 97%, 98% or 99% identity to SEQ ID NO: 61, or may comprise an polynucleotide sequence with at least 80%, 85%, 90%, 91%, 92%, 93%, 94%, 95%, 96%, 97%, 98% or 99% identity to SEQ ID NO: 62.

TGF-β expression within tumors is known to suppress the antitumor activity of leukocytes in the tumor microenvironment. Thus, in some embodiments, the modified NK-92 cell to be irradiated includes a recombinant nucleic acid construct that encodes a TGF-beta inhibitor, for example a peptide that inhibits TGF-β. In some embodiments, the nucleic acid construct encodes a TGF-beta trap. In some embodiments, the TGF-beta trap includes the extracellular domain of a TGFβRII molecule. In some embodiments, the TGF-beta trap includes a single chain dimer of the extracellular domain of a TGFβRII molecule, and most preferably includes a single chain dimer of the TGF-beta Receptor II ectodomain. In exemplary embodiments, the TGF-beta trap as contemplated herein may comprise a polynucleotide sequence with at least 80%, 85%, 90%, 91%, 92%, 93%, 94%, 95%, 96%, 97%, 98% or 99% identity to SEQ ID NO: 63 (TGFβRII extracellular domain), or SEQ ID NO: 65 (TGFb trap sequence). The TGF-beta trap contemplated herein may also comprise an amino acid sequence with at least 80%, 85%, 90%, 91%, 92%, 93%, 94%, 95%, 96%, 97%, 98% or 99% identity to SEQ ID NO: 64 (TGFβRII extracellular domain), or SEQ ID NO: 66 (TGFb trap sequence). Other suitable TGF-beta traps include those described in Mol. Canc. Ther. 2012, Vol 11(7), 1477-1487.

In some embodiments, the modified NK-92 cells to be irradiated as described herein are administered with a TGF-β inhibitor to block TGF-β and help remove immunosuppression. In some embodiments, the NK-92 cells described herein are administered with other immunotherapies to help decrease or eliminate a tumor. For example, TGF-β can be inhibited by intratumoral injection of inhibitory peptides in combination with intratumoral injections of poly(I:C) and an α-CD40 antibody. In some embodiments, the TGF-β inhibitor is combined with IL-2.

In additional embodiments, the irradiated recombinant NK-92 cells as described herein include a nucleic acid encoding a cytokine that provides selection for NK-92 cells that express the cytokine, such as IL-2, IL-12, IL-15, IL-18, or IL-21. In exemplary embodiments, the nucleic acid encodes a cytokine such as IL-2 or IL-15. In one embodiment, a cytokine, such as the IL-2 polypeptide may have a sequence with at least 80%, 85%, 90%, 91%, 92%, 93%, 94%, 95%, 96%, 97%, 98% or 99% identity to SEQ ID NO:42. In some embodiments, the IL-2 polypeptide may have a sequence with at least 80%, 85%, 90%, 91%, 92%, 93%, 94%, 95%, 96%, 97%, 98% or 99% identity to SEQ ID NO:43.

In one embodiment, the IL-2 is expressed with a signal sequence that directs the IL-2 to the endoplasmic reticulum IL-2 (“erIL-2”). In another embodiment, the IL-15 is expressed with a signal sequence that directs the IL-15 to the endoplasmic reticulum IL-15 (“erIL-15”).

Accordingly, in some embodiments, the cytotoxicity of the irradiated NK-92cells disclosed herein is dependent on the presence of cytokines (e.g., interleukin-2 (IL-2)). Thus, optionally, modified NK-92 cells are further modified to express at least one cytokine. Optionally, the at least one cytokine is IL-2, IL-12, IL-15, IL-18, IL-21 or a variant thereof. Optionally, the at least one cytokine is IL-2, IL-15 or a combination thereof. Optionally, the IL-2 and/or IL-15 is expressed with a signal sequence that directs the cytokine to the endoplasmic reticulum. Directing the IL-2 to the endoplasmic reticulum permits expression of IL-2 at levels sufficient for autocrine activation and without releasing substantial amounts of IL-2 extracellularly. Sec Konstantinidis et al “Targeting IL-2 to the endoplasmic reticulum confines autocrine growth stimulation to NK-92® cells” Exp Hematol. 2005 February;33(2):159-64. A representative nucleic acid encoding IL-2 is shown in SEQ ID NO: 14 and a representative polypeptide of IL-2 is shown in SEQ ID NO:15.

The irradiated modified NK-92 cells may comprise a nucleic acid sequence encoding IL-2, with 70%, 80%, 90%, or 95% identity to SEQ ID NO: 14. Optionally, the irradiated modified NK-92 cells may include a nucleic acid sequence with 90%, 91%, 92%, 93%, 94%, 95%, 96%, 97%, 98% or 99% identity to SEQ ID NO: 14. Optionally, the irradiated modified NK-92® cells may include a IL-2 polypeptide with 70%, 80%, 90%, or 95% identity to SEQ ID NO:15. Optionally, the irradiated modified NK-92 cells may include a IL-2 polypeptide with 90%, 91%, 92%, 93%, 94%, 95%, 96%, 97%, 98% or 99% identity to SEQ ID NO:15. The disclosed irradiated modified NK-92 cells advantageously are capable of being maintained in the absence of IL-2 without secreting IL-2 in an amount to cause a clinical adverse effect.

Furthermore, the nucleic acid construct (e.g., the quadricistronic vector) may also include a sequence that encodes a 2A peptide, such as a T2A, P2A, E2A, or F2A peptide, in order to produce equimolar levels of polypeptides encoded by the same mRNA. The E2A peptide contemplated herein may comprise a polynucleotide sequence with at least 80%, 85%, 90%, 91%, 92%, 93%, 94%, 95%, 96%, 97%, 98% or 99% identity to SEQ ID NO: 17. The T2A peptide as contemplated herein may comprise a polynucleotide sequence with at least 80%, 85%, 90%, 91%, 92%, 93%, 94%, 95%, 96%, 97%, 98% or 99% identity to SEQ ID NO: 18.

In one exemplary, non-limiting example, the nucleic acid construct (e.g., the quadcistronic vector) disclosed herein may include a polynucleotide sequence having at least 80%, 85%, 90%, 91%, 92%, 93%, 94%, 95%, 96%, 97%, 98% or 99% identity to SEQ ID NO: 19 (5′ Homology arm of AAVS1), SEQ ID NO:20 (EFla Promoter), SEQ ID NO: 21(T7 Promoter), SEQ ID NO: 22 (CCR7 cDNA), SEQ ID NO: 23 (P2A element), SEQ ID NO: 24 (IgHC leader), SEQ ID NO: 25 (CD19 CAR minus signal peptide), SEQ ID NO: 26 (high affinity CD16), SEQ ID NO: 27 (IRES), SEQ ID NO: 28(SC40 Poly-A), SEQ ID NO: 29 (3′ Homology arm of AAVS1), and/or SEQ ID NO: 30 (Homology arm of AAVS1) for targeting the AAVS1 locus (SEQ ID NO: 7).

In some embodiments, the modified NK-92 cells to be irradiated as described herein may include or also include a nucleic acid construct encoding an antigen binding protein (“ABP”). In some embodiments, the antigen binding protein specifically binds a tumor associated antigen. In some embodiments, the ABP includes a fragment of an antibody, such as an scFv. In some embodiments, the antigen binding protein includes or is part of a chimeric antigen receptor (CAR). In some embodiments, the nucleic acid encodes an ABP, a CAR, or an ABP that includes a CAR that specifically binds CD19, CD20, NKG2D ligands, CS1, GD2, CD138, EpCAM, HER-2, EBNA3C, GPA7, CD244, CA-125, MUC-1, ETA, MAGE, CEA, CD52, CD30, MUC5AC, c-Met, EGFR, FAP, WT-1, PSMA, NY-ESO1, CSPG-4, IGF1-R, Flt-3, CD276, CD123, PD-L1, BCMA, CD33, B7-H4, or 41BB.

Additionally or alternatively, the modified NK-92 cells to be irradiated as described herein encode for an antigen binding protein that binds an immune modulator protein in a tumor. Examples of immune modulator proteins found in tumors, include CTLA-4, PD-1, IDO-1, CD39, and CD73.

In exemplary embodiments, the modified NK-92 cells to be irradiated as described herein are transfected with a nucleic acid construct encoding one or more CAR molecules that specifically binds the programmed cell death ligand 1 (PD-L1) (e.g., an anti-PD-L1 CAR). For example, the nucleic acid construct encoding one or more CAR molecules that specifically binds PD-L1 has an amino acid sequence comprising at least 90%, 91%, 92%, 93%, 94%, 95%, 96%, 97%, 98% or 99% identical to SEQ ID NO:69 (which may be encoded by a nucleic acid sequence at least 90%, 91%, 92%, 93%, 94%, 95%, 96%, 97%, 98% or 99% identical to SEQ ID NO:68.

In specific embodiments, NK-92 cells are transfected with an expression vector (e.g., the quadricistronic vector) comprising: SEQ ID NO: 1 or a nucleic acid with 90%, 91%, 92%, 93%, 94%, 95%, 96%, 97%, 98% or 99% identity to SEQ ID NO:1; SEQ ID NO:25 (CD19 CAR) or a nucleic acid with 90%, 91%, 92%, 93%, 94%, 95%, 96%, 97%, 98% or 99% identity to SEQ ID NO:13 (CD16 F158V), or a nucleic acid or polypeptide sequence with 90%, 91%, 92%, 93%, 94%, 95%, 96%, 97%, 98% or 99% identity to SEQ ID NO: 14 (erIL-2 n.t. sequence); and/or SEQ ID NO: 15 (erIL-2 a.a. sequence), or a nucleic acid with 90%, 91%, 92%, 93%, 94%, 95%, 96%, 97%, 98% or 99% identity to SEQ ID NO:14.

In other specific embodiments, NK-92 cells are transfected with an expression vector (e.g., the quadricistronic vector) comprising: SEQ ID NO:47 (CXCR2) or a nucleic acid with 90%, 91%, 92%, 93%, 94%, 95%, 96%, 97%, 98% or 99% identity to SEQ ID NO:47; SEQ ID NO:25 (CD19 CAR) or a nucleic acid with 90%, 91%, 92%, 93%, 94%, 95%, 96%, 97%, 98% or 99% identity to SEQ ID NO:12; and SEQ ID NO:13 (CD16 158V), and/or a nucleic acid with 90%, 91%, 92%, 93%, 94%, 95%, 96%, 97%, 98% or 99% identity to SEQ ID NO: 14; and/or SEQ ID NO:15 (erIL-2), or a nucleic acid with 90%, 91%, 92%, 93%, 94%, 95%, 96%, 97%, 98% or 99% identity to SEQ ID NO:14. Suitable expression vectors are known in the art and can be used. In further aspects, the recombinant nucleic acid comprises a segment encoding erIL-15, and the nucleic acid encoding erIL-15 has at least 90%, 91%, 92%, 93%, 94%, 95%, 96%, 97%, 98% or 99% identity to SEQ ID NO:67. Optionally, the expression vector is a plasmid.

In an optional embodiment, the CAR may comprise a CD19CAR_CD3a having a polynucleotide sequence having at least 80%, 85%, 90%, 91%, 92%, 93%, 94%, 95%, 96%, 97%, 98% or 99% identity to SEQ ID NO: 41.

Considered form a different perspective, the irradiated recombinant NK-92 cells having maintained activity can be administered to cancer patients for decreasing cancer cells or tumor size. Accordingly, methods for treating a cancer or a tumor in a subject include administering to the subject a therapeutically effective amount of an irradiated recombinant NK-92 cell. First the wherein the recombinant NK-92 cell is transfected with a nucleic acid as disclosed herein encoding i) a homing receptor; ii) an antigen binding protein (ABP) or a chimeric antigen receptor (CAR) that specifically binds a target antigen; iii) an Fc Receptor; and/or iv) a secreted immune modulator selected from a TGFβ inhibitor and/or IL-12, wherein the nucleic acid is operably linked to a promoter. The transfected recombinant NK-92 cell is subsequently irradiated with 2.5, 5, 10, or 15 Gray. The irradiated recombinant KN-92 cells are subsequently administered to the subject (e.g., cancer patient). In some embodiments, about 1×10³ to 1×10¹⁰, per m² of the irradiated recombinant NK-92 cells are administered to the subject. Administration of the irradiated recombinant NK-92 cells as disclosed herein, may be parenterally, intravenously, peritumorally, or infused into the subject.

According to the methods provided herein, the subject is administered an effective amount of the irradiated recombinant NK cells as disclosed herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

Pharmaceutically acceptable compositions can include a variety of carriers and excipients. A variety of aqueous carriers can be used, e.g., buffered saline and the like. These solutions are sterile and generally free of undesirable matter. Suitable carriers and their formulations are described in Remington: The Science and Practice of Pharmacy, 22nd Edition, Loyd V. Allen et al., editors, Pharmaceutical Press (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

### Kits

Provided herein are kits comprising the recombinant NK-92 cells described herein. In some embodiments, the kit comprises recombinant NK-92 cells comprising one or more nucleic acid sequences encoding i) a homing receptor, ii) an ABP or CAR that specifically binds to a target antigen, iii) an Fc Receptor and/or iv) a secreted immune modulator selected from a TGFβ inhibitor and/or IL-12. Optionally, one or more proteins encoded by the nucleic acid sequences are expressed on the cell surface of the recombinant NK-92 cells cells. In some embodiments, kit comprises a recombinant NK-92 cell comprising a nucleic acid encoding C-C chemokine receptor type 7 (CCR7), CXCR2, or the receptor for CXCL14 operably linked to a promoter. Optionally, the nucleic acid encoding CCR7 has at least 90%, 91%, 92%, 93%, 94%, 95%, 96%, 97%, 98% or 99% identity to SEQ ID NO:1. Optionally, the homing receptor is expressed on the cell surface of the recombinant NK-92 cells. Optionally, the promoter comprises one or more NFAT binding elements and a minimal promoter. Optionally, the promoter has at least 90%, 91%, 92%, 93%, 94%, 95%, 96%, 97%, 98% or 99% identity to SEQ ID NO:6. Optionally, one or more proteins encoded by the nucleic acid sequences are expressed on the cell surface of the recombinant NK-92 cells.

Optionally, the recombinant NK-92 cells are provided in a composition comprising a pharmaceutically acceptable excipient. Optionally, a kit may contain additional compounds such as therapeutically active compounds or drugs that are to be administered before, at the same time or after administration of the recombinant NK-92 cells. Optionally, instructions for use of the kits will include directions to use the kit components in the treatment of a cancer. The instructions may further contain information regarding how to prepare (e.g., dilute or reconstitute, in the case of freeze-dried protein) the antibody and the recombinant NK-92 cells (e.g., thawing and/or culturing). The instructions may further include guidance regarding the dosage and frequency of administration.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

### EXAMPLES

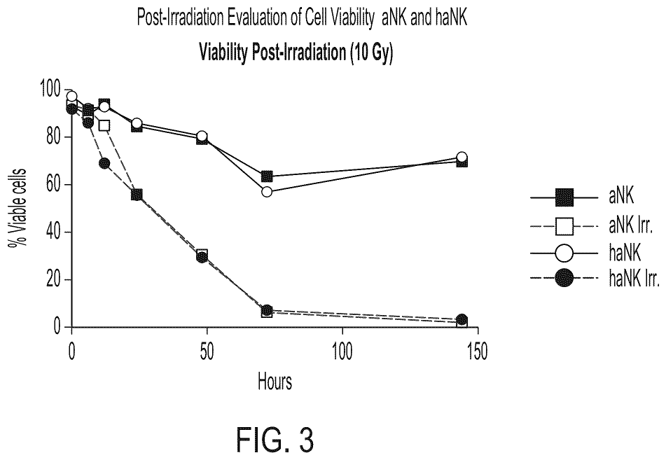

With reference to FIGS. 2A-2B, wild type NK-92 cells were irradiated with radiation doses ranging from 0 to 30 Gy and the cell concentration and percent viability were measured accordingly over 5 days (120 hours). Cell concentration and percent viability in irradiated (10 Gy) and non-irradiated aNK and haNK cells are shown in FIG. 3.

The PD-L1 t-haNK cell line has been developed for administration to cancer patients with tumors that express the PD-L1 antigen and may also be used in combination regimens with antibodies that recognize tumor-specific antigens. A PD-L1 t-haNK cell line secreting a TGFβ antagonist (TGFbeta-trap/PD-L1 t-haNK) by transfection with a quadricistronic vector is schematically depicted in FIG. 4A was developed for blocking TGFbeta activity in the tumor. However, allogeneic cells can induce harmful effects when infused in cancer patients. Accordingly, to avoid these deleterious effects the TGFbeta-trap/PD-L1 t-haNK cells were irradiated prior to infusion. Considering a higher irradiation dose may also negatively influence the cell function and in particular the secretion of the TGFβ, an optimal irradiation dose is highly desired for inhibiting proliferative capacity without affecting cell function. Accordingly, the cell concentration of TGFbeta-trap/PD-L1 t-haNK cells after irradiation is shown in FIG. 4B, while secretion of the TGFbeta-trap molecule (assaying of TGFbeta-RII ectodomain) after irradiation is shown in FIG. 4C.

In another embodiment, a quadricistronic vector for expressing IL-12 in a PD-L1 t-haNK cell line (IL-12/PD-L1 t-haNK) is schematically depicted in FIG. 4D. In some embodiments, administration of irradiated PD-L1 t-haNK cells into a subject confer inhibition of IL-12 activity in cancer cells and/or tumor cells in the subject.

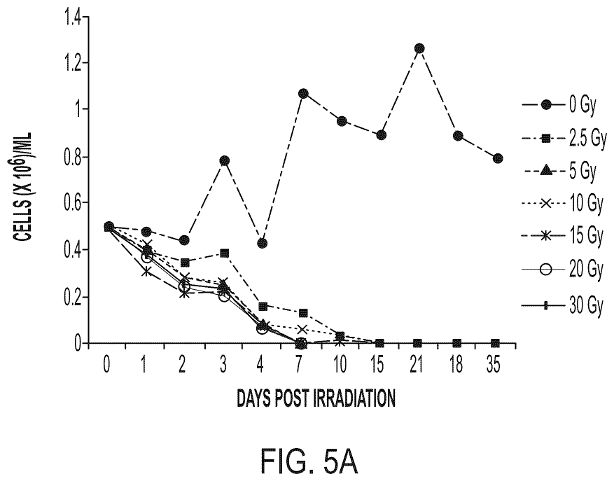

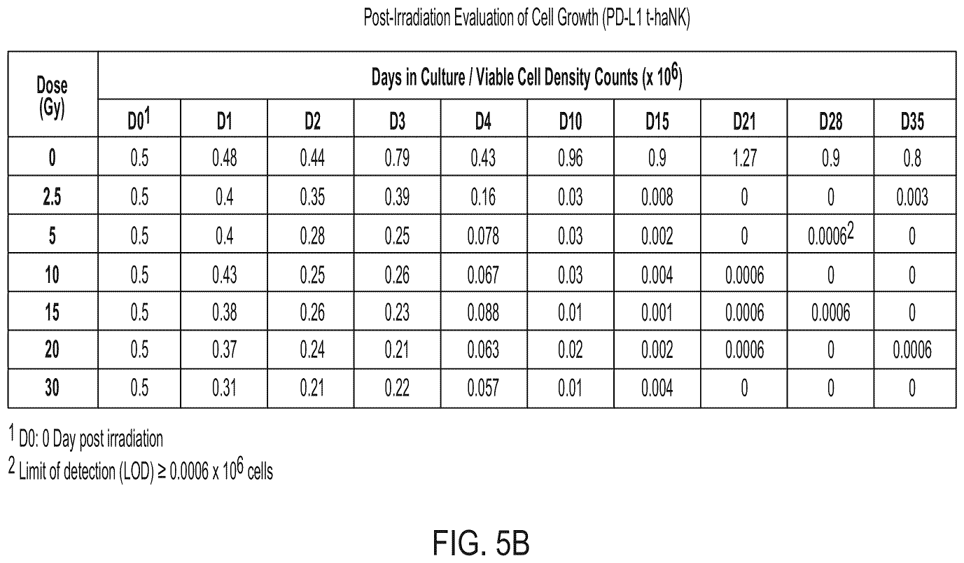

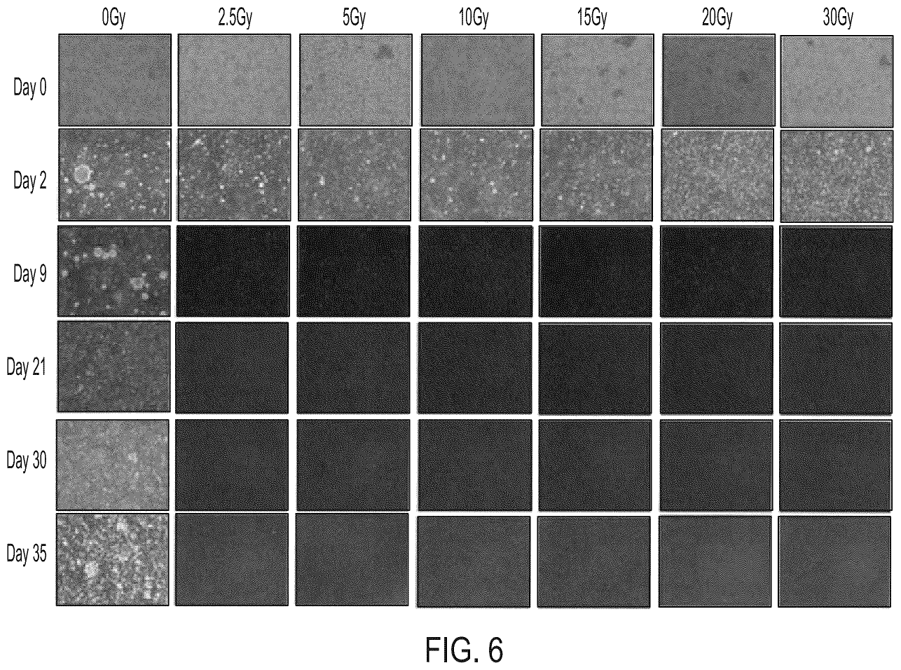

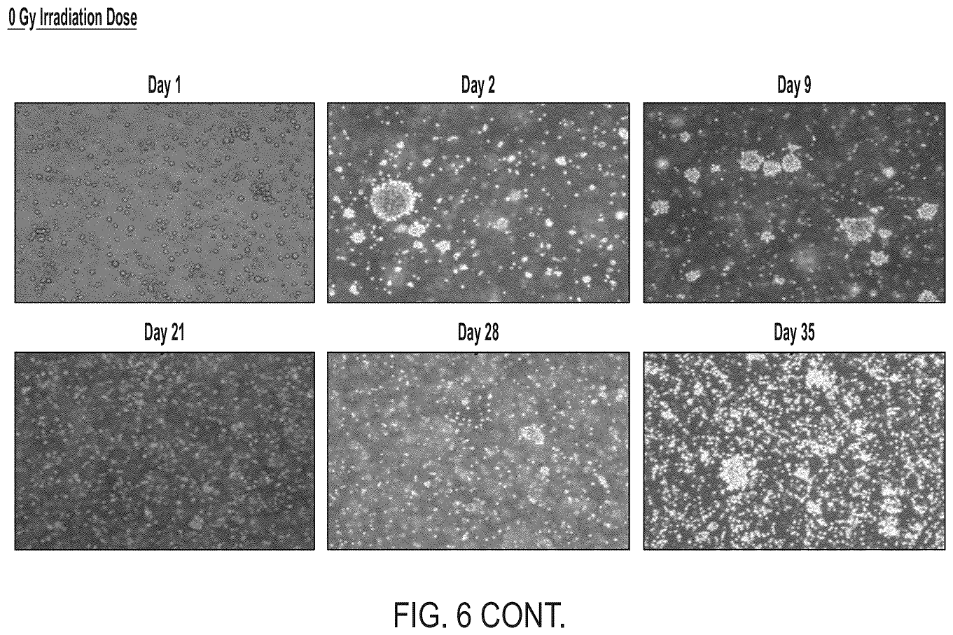

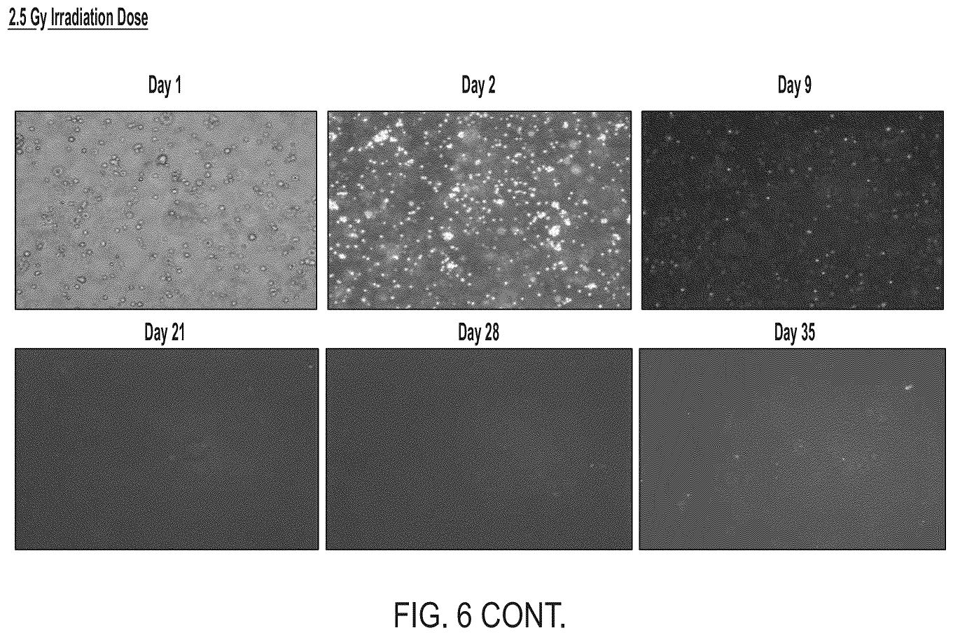

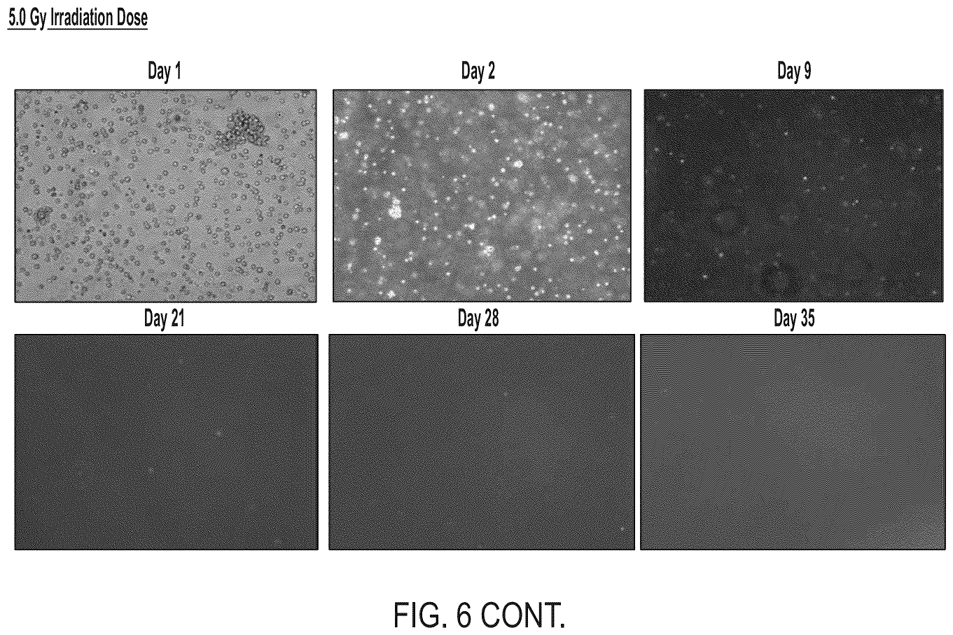

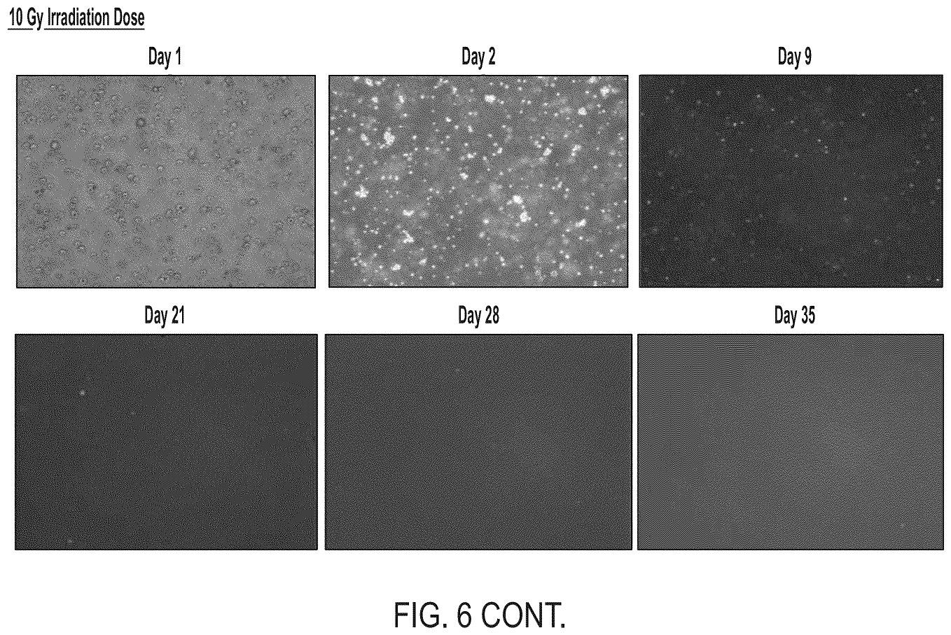

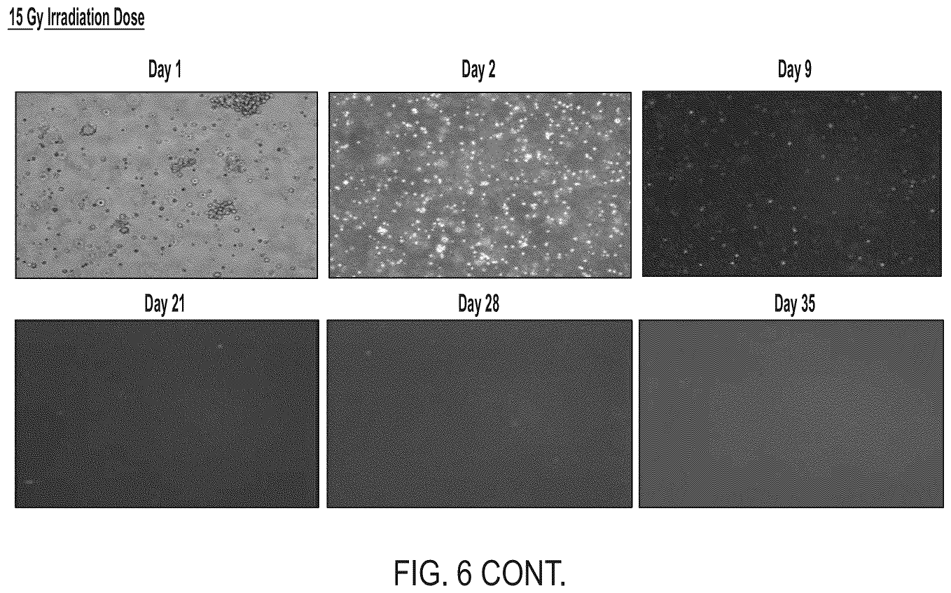

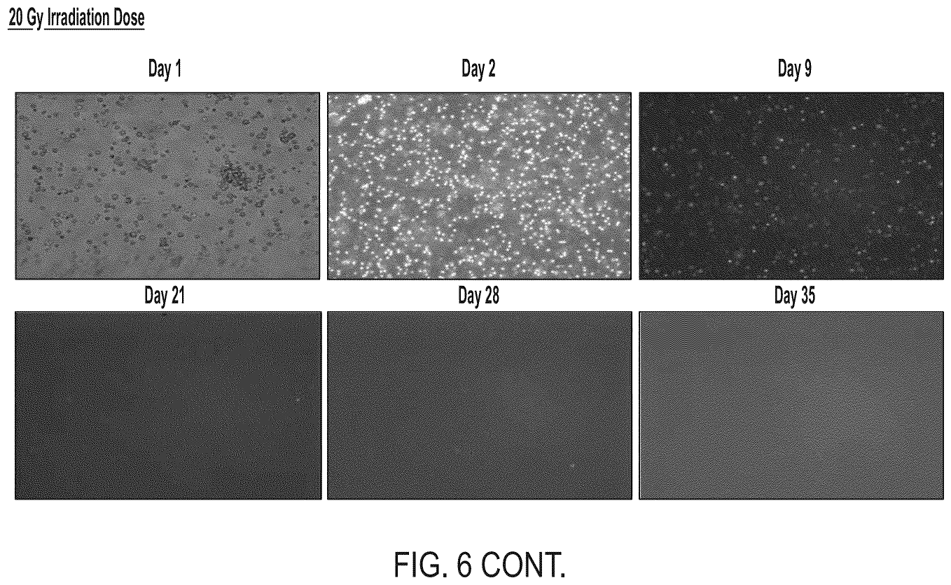

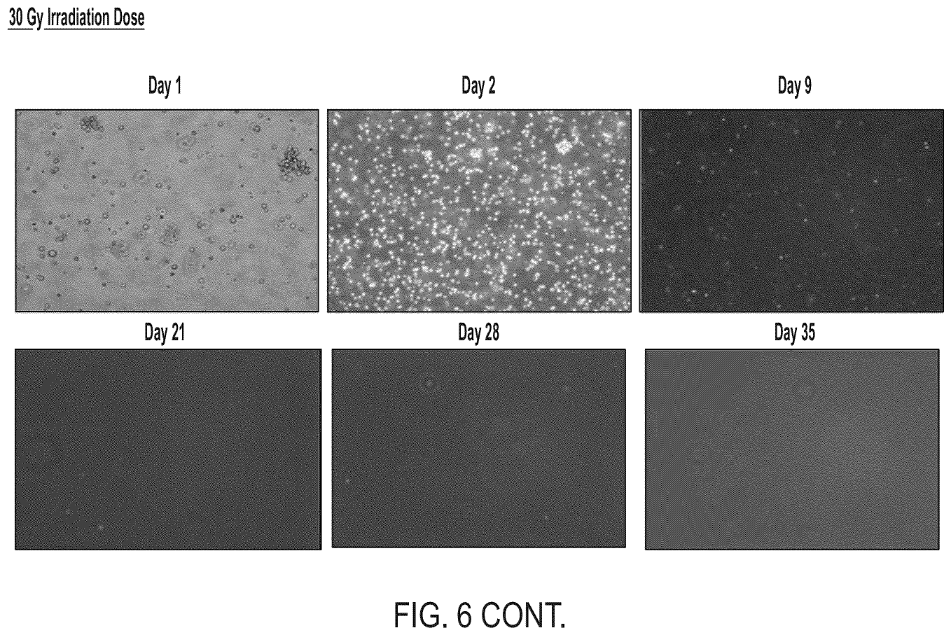

With reference to FIGS. 5A-5B, PD-L1 t-haNK cells were subjected to increasing doses of X-ray irradiation using an RS-2000 Irradiator. After irradiation, cells were set for long-term culture under optimal growth conditions using complete growth media. Viable cells were assessed regularly using a variety of methods including cell density (FIGS. 5A and 5B), cell health assays (FIGS. 7A and 7B) and imaging cytometry methods (FIGS. 6, 7A, 7B) for a period of 35 days.

### Study Design

PD-L1 t-haNK Cells were formulated in 5% Albumin (Human) at 4.0×10⁷ cells/mL and irradiated using a RS-2000 irradiator at levels of 0, 2.5, 5, 10, 15, 20 and 30 Gy. Immediately following irradiation, cells were diluted in complete growth media (cGM) and placed in an incubator maintained at 37° ° C. with 5% CO₂ atmosphere. Non-irradiated (0 Gy) cells were used as a control. Analytical testing was performed on alternate days during the first week of study followed by weekly testing throughout the remainder of the study. Cell cultures were replenished with fresh media (two-thirds volume) every week.

PD-L1 t-haNK Cell Concentration and Irradiation

PD-L1 t-haNK cells were concentrated by centrifugation at 336×g for 5 minutes. Cell pellet was suspended in 5% Albumin (human) USP and the concentration was adjusted to 4.0×10⁷ cells/mL. The concentrated cells in different T-25 flasks were irradiated at room temperature with different doses of X-ray irradiation using the RadSource RS-2000. The machine was programmed to irradiate the cells at specified doses and the irradiation time was automatically set by the machine (Table 1).

TABLE 1
IRRADIATION TIME VS DOSE

| SHELF | IRRADIATION DOSE (GY) | TIME TAKEN TO IRRADIATE |
|---|---|---|
| 3 | 0 | 0 |
| | 2.5 | 1 MIN 39 SEC |
| | 5 | 3 MIN 17 SEC |
| | 10 | 3 MIN 31 SEC |
| | 15 | 5 MIN 17 SEC |
| | 20 | 7 MIN 22 SEC |
| | 30 | 10 MIN 33 SEC |

### Analytical Testing

To evaluate the impact of irradiation on cell proliferative capacity, cell health (cell vitality) and viability was monitored over 35 days of culture. With reference to FIGS. 6, 7A, and 7B, samples were taken from the flasks on Days 1, 2, 3, 4, 7, 10, 15, 21, 28 and 35 and analytical testing was performed to measure; (a) cell viability and viable cell count by CCV NC-200 test method; (b) cell morphology by bright field microscopy and (c) cell health using NucleoCounter® NC3000™ image cytometer. Nonirradiated cells were used as study control.

PD-L1 t-haNK Cell Growth Analysis Post Irradiation

Cell viability and viable cell counts were determined by CCV NC-200 test method. In the flask containing the irradiated cells, the viable cell density (VCD) of the cells significantly decreased from the initial 5×10⁵ cells/mL by day 7 of culture while the numbers in non-irradiated flask showed a rapid outgrowth within seven days (FIGS. 5A-5B). No cell growth was observed in any of the flasks containing cells irradiated with approximately 5 Gy dose of irradiation for up to 35 days.

Cell counts by CCV NC-200 test method were further confirmed by brightfield imaging using EVOS microscope. Visualization of cell morphology during the culture period confirmed a significant decline in cell numbers in irradiated flasks by day 9 (FIG. 6) and no live cells were observed from day 15 till the end of the study.

In the Annexin assay, cells are stained using fluorescently labeled Annexin V and Propidium Iodide (PI), the latter to distinguish between the early and late apoptotic/necrotic cells. Culture cell health was evaluated by Annexin V staining using the imaging cytometry by NucleoCounter® NC3000™. Annexin V staining in combination with Hoeschst 33342 and Propidium Iodide (PI) showed an increase in number of apoptotic (Annexin+PI+, upper right quadrants) and pre-apoptotic (Annexin+PI-, lower right quadrants) cells after two days of culture in flasks irradiated with 2.5 Gy dose of irradiation and above (FIG. 7A).

PD-L1 t-haNK Cell Health Evaluation Using Vitality Assay

Cell vitality was measured by detecting changes in levels of intracellular reduced thiols (glutathione; GSH), which is an early hallmark of cells death progression. The cell health in culture was evaluated by Vitality Assay using the imaging cytometry by NucleoCounter® NC3000™. A significant decrease in cellular GSH concentration (GSH+, lower right quadrants) and increase in PI positive dead cells (upper quadrants) was observed in flasks irradiated with 2.5 Gy dose of irradiation and above after two days of culture (FIG. 7B).

The objectives of this study were to assess the effect of different doses of X-ray irradiation on proliferative capacity of PD-L1 t-haNK cells. These results collectively demonstrate that RS-2000 irradiator programmed at 15 Gy dose can prevent proliferative capacity of PD-L1 t-haNK cells. However, no cell growth was observed when cultures were irradiated in the range of 5 to 30 Gy X-ray irradiation dose. The non-irradiated cells (control sample) were able to grow in culture for up to 35 days.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a cancer or a tumor in a subject, the method comprising:
administering to the subject a therapeutically effective amount of irradiated recombinant NK-92 cells, wherein the irradiated recombinant NK-92 cells are transfected with one or more nucleic acids that encode
i) a homing receptor;
ii) an antigen binding protein (ABP) or a chimeric antigen receptor (CAR) that specifically binds a target antigen;
iii) an Fc Receptor; and
iv) a secreted immune modulator selected from a TGFβ inhibitor and/or IL-12,
wherein the nucleic acid is operably linked to a promoter, and wherein the transfected recombinant NK-92 cell is irradiated with 2.5 to 15 Gy; and
administering the irradiated transfected recombinant NK-92 cell to the subject.

2. The method of claim 1, wherein the recombinant NK-92 cells are transfected with a vector.

3. The method of claim 1, wherein the recombinant NK-92 cells are transfected with a quadri-cistronic recombinant nucleic acid.

4. The method of claim 1, wherein the homing receptor is a G protein-coupled receptor, a cytokine receptor, a selectin, or an integrin.

5. The method of claim 1, wherein the homing receptor is selected from the group consisting of CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCXCKR, D6, DARC, or a receptor for CXCL14.

6. The method of claim 1, wherein the nucleic acid encodes the antigen binding protein.

7. The method of claim 6, wherein the ABP is selected from a protein or antibody that binds to CD19, CD20, GD2, HER-2, CD30, EGFR, FAP, CD33, CD123, PD-L1, IGFIR, CSPG4, or B7-H4.

8. The method of claim 6, wherein the ABP is selected from a protein or antibody that binds to PD-L1.

9. The method of claim 1, wherein the nucleic acid encodes the CAR.

10. The method of claim 1, wherein the CAR specifically binds PD-L1.

11. The method of claim 1, wherein the Fc Receptor is CD16 or a high affinity CD16.

12. The method of claim 1, wherein the secreted immune modulator is IL-12.

13. The method of claim 12, wherein the IL-12 is a single chain IL-12.

14. The method of claim 1, wherein the secreted immune modulator is a TGFβ inhibitor.

15. The method of claim 1, wherein the TGFβ inhibitor comprises a single chain dimer of a TGF-beta Receptor II ectodomain.

16. The method of claim 1, wherein from 1×10³ to 1×10¹⁰, per m² of the irradiated recombinant NK-92 cells are administered to the subject.

17. The method of claim 1, wherein the recombinant NK-92 cells are administered parenterally, intravenously, peritumorally, or by infusion.

18. The method of claim 1, further comprising administering to the subject a therapeutic drug.

19. The method of claim 1, wherein the recombinant NK-92 cells, after irradiation, maintain transient activity of the secreted immune modulator.

20. The method of claim 19, wherein the recombinant NK-92 cells, after irradiation, are inhibited from replication.
